(19) United States
(12) Patent Application Publication
(10) Pub. No.: US 2024/0277888 A1
(43) Pub. Date: Aug. 22, 2024

(54) BIODEGRADABLE AND REUSABLE CELLULOSIC MICROPOROUS SUPERABSORBENT MATERIALS

(71) Applicants: CELLUCOMP LIMITED, Lothian (GB); THE JAMES HUTTON INSTITUTE, Dundee (GB)

(72) Inventors: Eric Arron WHALE, Lothian (GB); David Gwyddon HEPWORTH, Lothian (GB); Andrew John LOVE, Dundee (GB); Julie Nicola SQUIRES, Dundee (GB)

(73) Assignees: CELLUCOMP LIMITED, Lothian (GB); THE JAMES HUTTON INSTITUTE, Dundee (GB)

(21) Appl. No.: 18/580,652

(22) PCT Filed: Jul. 20, 2022
(86) PCT No.: PCT/EP2022/070401

§ 371 Date: Jan. 19, 2024

(30) Foreign Application Priority Data
Jul. 20, 2021 (GB) 2110423.7
Jul. 20, 2021 (GB) 2110426.0

(51) Int. Cl. A61L 15/60; A61L 15/42; C08L 97/02

(52) U.S. Cl. CPC A61L 15/60; A61L 15/425; C08L 97/02; A61L 2300/404

(57) ABSTRACT

The present invention relates to a process for preparing an antimicrobial cellulose-containing microporous superabsorbent composition from an herbaceous plant material, the process comprising the step of comminuting dry granulated herbaceous plant material to form microparticles having an average particle diameter of from 100 μm to 800 μm; to obtain the cellulose-containing microporous superabsorbent composition.

### TECHNICAL FIELD

The present invention relates to a process for preparing a biodegradable and reusable cellulose-containing adsorbent materials, with tuneable morphology and inherent antimicrobial activity, from herbaceous plant material. The materials are useful for a wide variety of applications, such as air filtration; water filtration; garments; surgical bandages and packing materials, and generally, any application where the ability adsorb and desorb humidity and stop bacterial growth and/or viral proliferation is desired. The invention also relates to methods and compositions for materials having a non-leaching component that imparts antimicrobial properties. The modification may be applied to cellulose-containing adsorbent materials such as particulate cellulose-containing adsorbents, or films prepared therefrom, resulting in essentially non-leaching modification of substrates, which impart to the absorbent a high antimicrobial efficacy.

### BACKGROUND TO THE INVENTION

The present invention relates generally to the field of biodegradable cellulosic adsorbent materials, more specifically to highly adsorbent materials with added inherent antimicrobial activity, which can be reused after drying off of volatile fluids, and their use in a variety of different applications, including materials for air filtration; water filtration; garments; surgical bandages and packing materials, and generally, any application where the ability to adsorb and desorb humidity and stop bacterial growth and/or viral proliferation is desired. US2019/202940 A1 concerns a method for preparing cellulose-containing material comprising the steps of treating plant material with a peroxide agent and water, allowing the mixture to hydrate until the pH of the mixture is pH 4.5 or less and homogenising this wet, peroxide-treated material. GB 2145103 A concerns a process for preparing absorbent materials from a pectin-containing vegetable material. The process comprises the steps of: (i) comminuting the vegetable material to a particle size of from 0.05 mm to 3.0 μmm; (ii) de-esterifying the pectin to a degree of esterification of less than 45%; (iii) washing the vegetable material in soft water; and (iv) drying the vegetable material to a moisture content of less than 15%. The specific example from this patent involve comminuting wet material. It is known in the art that antimicrobial agents can enhance the antimicrobial activity of a material. These include metal modification, in particular by copper or silver deposition, which may exhibit strong antimicrobial properties. Also known are antimicrobial peptides, e.g. proteolytic enzymes that can attack and destroy bacteria, fungi or virus. However, the modification of absorbent materials has not been very successful, and usually results in leaching, or loss of activity upon washing and/or drying. The known prior art processes for formation of metal nanoparticles are usually complex, often requiring high temperature treatment and as such there is a need for much simpler methods.

A main function of absorbents is the absorption of various fluids. These fluids, e.g. wound exudates, are frequently rich in nutrients and are capable of supporting abundant bacterial growth, which can easily cause serious infection and may also release a variety of harmful toxins. Filter materials may also acquire a number of biological pathogens, such as microbes and viral particles by physical adsorption to the surface thereto, but once the surface is saturated, these may be released and leach out into the medium. This is in particular relevant for filters that are to be reused, e.g. filter materials for breathing air, e.g. face masks or HVAC filters for vehicles or buildings, wherein the filters traditionally have been replaced once fully loaded.

Unfortunately, it had proven difficult to produce an effective disinfectant that does not readily wash out of the material, in particular when subjected to an industrial our household detergents and washing and drying process. Hence wash out, or leaching usually occurs, which reduces effectiveness and may cause irritation, or infections. This is a general problem where absorptive packings are placed in contact with significant possibility for dangerous pathogen load, e.g. bacterial growth. When used for care products, particularly tampons, the well-known “toxic shock syndrome” results from multiplication of Staphylococcus aureus bacteria.

In addition, due to the globalization of transport, the emergence of new diseases and pandemics, the uses for a technology that imparts an essentially non-leaching re-useable antimicrobial modification to a variety of materials is duly recognized. Specific areas of use are described below herein. And last but not least, sustainable materials would be desired which may simply be subjected to compost preparations, preferably after extraction of valuable antimicrobial components, metals and the like.

In conclusion, with regard to the above descriptions of the art, it is apparent that there is a need for an improved adsorbent that has enhanced antimicrobial abilities, is superabsorbent, and can be modified or shaped easily, offers antimicrobial activity and is non-leaching upon use, washable and reusable, and that allows recycling at the end of life due to entirely sustainable components.

Accordingly, applicants have found that absorbent materials that are easy to prepare, recyclable and reusable, and also a simple method to impart antimicrobial properties to these materials and their applications ranging from healthcare applications, filters, water sterilization, to garment applications.

### BRIEF DESCRIPTION OF THE DRAWINGS

Embodiments of the invention are further described hereinafter with reference to the accompanying drawings, in which:

FIG. 1 is SEM images of the materials prepared according to Example 1 (top), Example 2 (middle) and Example 3 (bottom).

FIG. 2 is a UV-VIS spectrum of the supernatant used to wash silver metallised (AgNP) cellulose-containing microporous superabsorbent composition of Example 7 at 70° C. for 120 μminutes, of wash cycles 1 (29-3 CurranAgW1), wash cycle 2 (29-3 CurranAgW2) and wash cycle 4 (29-3 CurranAgW4).

FIG. 3 is a cartoon explaining what materials were placed on an existing culture of bacteria on an agar plate or what materials were placed on an agar plate after which bacteria were allowed to grow (see FIGS. 4 and 5).

FIG. 4 are images showing experimental results of GFP-expressing Escherichia coli EC5025 bacteria after 24 h contact with the composition according to the invention compared to controls. All the plates are deposited in an identical pattern as depicted for 4 A (as also explained by the cartoon of FIG. 3) and are orientated in the same direction.

FIG. 5 are images showing experimental results of GFP-expressing Pseudomonas syringae KP71 bacteria after 24 h contact with the composition according to the invention compared to controls. All the plates are deposited in an identical pattern as depicted for 5 A (as also explained by the cartoon of FIG. 3) and are orientated in the same direction.

FIG. 6 is a photograph of a slurry of material prepared according to the method of Example 15, where GFP-SpyCatcher was attached to functionalised cellulose particulate material according to Example 12, varying the amount of GFP-SpyCatcher used. The mixture was washed several times in PBS to remove unbound components. From FIG. 6 it can be observed that GFP-SpyCatcher is attached to the cellulose particulate material. The SpyCatcher motif contains a Histidine-tag for convenient analysis by for example standard Western Blotting techniques. From left to right we see the results of using a 25 μg/mL GFP (fluorescing), 50 μg/mL GFP (fluorescing more brightly), 100 μg/mL GFP (fluorescing more brightly still), 200 μg/mL GFP (fluorescing most brightly) and functionalised cellulose particulate material not contacted with GFP-SpyCatcher (not fluorescing).

FIG. 7 is a photograph of a Western Blot, wherein Dispersin B (DSPB) or metal binding tagged Dispersin B (AgDSPB) are confirmed to be attached to cellulose particulate material or antimicrobial cellulose-containing microporous superabsorbent composition (−/+silver metallisation) according to the invention and after three times washing in PBS. DSPB and AgDSPB contain a histidine tag, antibodies to which were used to detect the DSPB and AgDSPB on the Western Blot. DSPB can be bound to the cellulose particulate material at a ratio of about 1 μg/10 μmm². Lanes labelled with 1 indicate ladder. Lanes 2 to 6 indicate cellulose material according to the invention, incubated for 16 h with PBS (lane 2), incubated for 4 h with DSPB (lane 3), incubated for 16 h with DSPB (lane 4), incubated for 4 h with AgDSPB (lane 5), incubated for 16 h with AgDSPB (lane 6). Lanes 7 to 11 indicate silver metallised cellulose material according to the invention, incubated for 4 h with DSPB (lane 7), incubated for 16 h with DSPB (lane 8), incubated for 16 h with PBS (lane 9), incubated for 4 h with AgDSPB (lane 10), incubated for 16 h with AgDSPB (lane 11). Control lanes 12 to 17 indicate DSPB (lanes 12 to 14) or AgDSPB (lanes 15 to 17) at from left to right decreasing concentrations of DSPB or AgDSPB.

FIG. 8 is a graph of the results of Example 14, wherein invertase was functionally assessed after attachment to the material according to the invention. See Example 14 for more details.

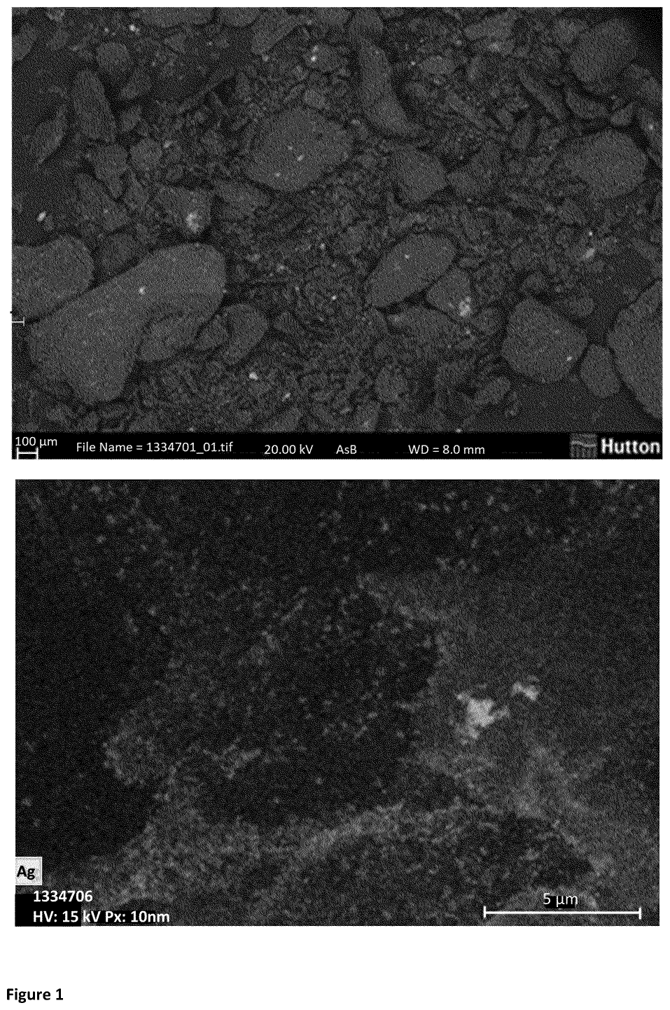

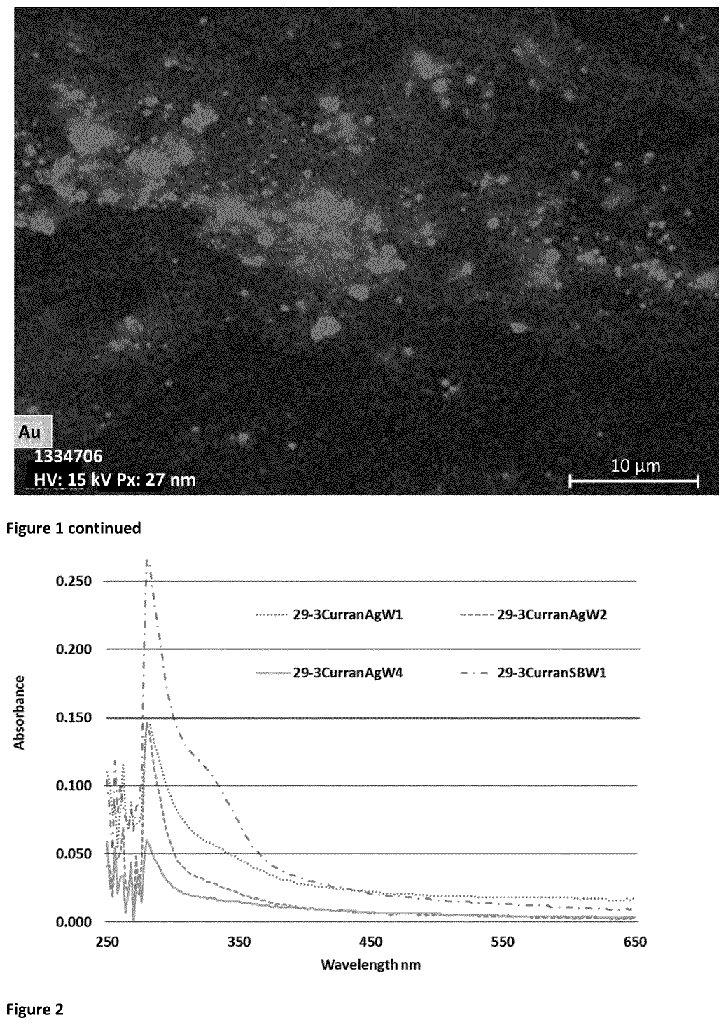

### SUMMARY OF THE INVENTION

In a first aspect, the present invention relates to a process for preparing an antimicrobial cellulose-containing microporous superabsorbent composition from an herbaceous plant material, the process comprising the steps of:
(a) comminuting dry granulated herbaceous plant material to form microparticles having an average particle diameter of from 100 μm to 800 μm;
to obtain the cellulose-containing microporous superabsorbent composition.

Applicants surprisingly was found that the absorbent materials as such has an inherent antiviral activity, at least initially before being wetted, and to delay growth of in particular enveloped viruses for a certain period of time. However, this activity can be exponentially improved by modification with an antimicrobial agent.

Surprisingly, the present process finds several unexpected advantages despite commencing with particles of plant material of a size broadly equivalent to that obtained in prior art processes which homogenise plant material in water to form a slurry. The advantages noted include a viscosity for the present slurry obtained in step (a) which allows improved processing at a higher solids content relative to prior art processes. Also, it has surprisingly been found that forming the plant material into the particles without complete degradation of the cell wall enables the material to form a superabsorbent material, that can be readily dried and reused, and that exhibit antimicrobial and in particular antiviral properties.

In a second aspect, the present invention provides cellulose-containing superabsorbent material obtainable by the process of the present invention, the cellulose-containing material having a fluid-superabsorbent volume area able to absorb of from 2 to 10 times of the original weight of water within 30 seconds (WAC), and exhibiting a virucidal activity as expressed by a reduction in viral titre of influenza A and/or human coronavirus of above 90%, as determined pursuant to standard method ISO18184:2019.

### DETAILED DESCRIPTION OF THE INVENTION

In a first aspect, the present invention relates to a process for preparing an antimicrobial cellulose-containing microporous superabsorbent composition from an herbaceous plant material, the process comprising the steps of:
(a) comminuting dry granulated herbaceous plant material to form microparticles having an average particle diameter of from 100 μm to 800 μm;
to obtain the cellulose-containing microporous superabsorbent composition.

Surprisingly, the present process finds several unexpected advantages despite commencing with particles of plant material of a size broadly equivalent to that obtained in prior art processes which homogenise plant material in water to form a slurry. The advantages noted include a viscosity for the present slurry obtained in step (a) which allows improved processing at a higher solids content relative to prior art processes. Also, it has surprisingly been found that forming the plant material into the particles without complete degradation of the cell wall enables the material to form a superabsorbent material, that can be readily dried and reused, and that exhibit antimicrobial and in particular antiviral properties.

Preferably, the process comprises the steps of: (a) comminuting dry granulated herbaceous plant material to form microparticles having an average particle diameter of from 100 μm to 800 μm; (b) optionally contacting the microparticles with an aqueous solution; preferably comprising an alkaline reagent, to wash out soluble components; removing the aqueous solution from the washed microparticles; and also optionally, drying the microparticles, and (c) optionally, forming a film and allowing the film to dry; and (d) at least once contacting the microparticles or the microparticle film with an antimicrobial agent precursor under conditions inductive of the formation, attachment or binding of an antimicrobial agent, and (e) isolating the antimicrobially modified microporous superabsorbent composition or the antimicrobially modified microporous film.

Applicants surprisingly found that the absorbent materials as such has an inherent antiviral activity, at least initially before being wetted, and to delay growth, of in particular enveloped viruses for a certain period of time. However, this activity can be exponentially improved by modification with an antimicrobial agent.

A number of antimicrobial materials based on leaching of low concentrations of silver ion from surfaces have also been reported, see for instance U.S. Pat. Nos. 6,126,931 and 6,030,632 describing biguanide polymers bonded a substrate, and subsequent bonding of silver salts to the immobilized biguanide polymer. The surface-bound biguanide polymer alone is shown as not inhibiting bacterial growth, but to adsorb the bacteria, thereby allowing the low-solubility silver salts to function as bactericide. A similar invention is reported in U.S. Pat. No. 5,662,913, disclosing wound dressings comprising silver salts stabilized by polyether polymers, or U.S. Pat. No. 5,856,248, using copper instead of silver salts. U.S. Pat. No. 5,985,301 discloses cellulose fibres that contain silver as an antibacterial agent; herein, in short, cellulose is dissolved in a solvent, and then silver compounds are added. Fibres spun from the solutions were found to impart bactericidal properties. The publication however indicates that the thus obtained materials enhance antibacterial effects by promoting the discharge of silver ions from the silver-based antibacterial agent, i.e. allow leaching. However, neither the polyether nor the biguanide polymers are available directly from naturally occurring products, and are not easily digested or otherwise recyclable.

In order to enhance the function of cellulose-containing products, various peptides and proteins can also advantageously be attached thereto. For example, peptides or proteins bound to cellulose formed into a paper sheet are used in the industry for screening or diagnostic purposes by screening for peptide-protein interactions or enzymatic (coloured) reactions. In the medical and hygiene industries the disinfective properties of cellulose-containing materials such as wound-dressings, face masks and garments, can be enhanced by the attachment of antimicrobial peptides or proteins (AMP). These are just but a few examples of the myriad of uses of cellulose-peptide/protein compositions.

A common method of attaching proteins or enzymes to cellulose is the 1-Cyano-4-dimethylaminopyridinium tetrafluoroborate (CDAP) method, which is an expensive and time-consuming method. Prior to the attachment of peptides or proteins the cellulose can be treated to enhance the attachment. TEMPO ((2,2,6,6-tetramethylpiperidin-1-yl)oxyl)-oxidized cellulose nanofibers have been used as a cellulose material upon which peptides or proteins are immobilized via electrostatic interactions or covalent immobilization (TEMPO-Oxidized Nanofibrillated Cellulose as a High Density Carrier for Bioactive Molecules 13 Weishaupt et al, 2015). AMPs have also been immobilized on TEMPO-oxidized cellulose nanofibers after for example functionalization of the fibers with alkyl ketene dimer (Immobilization of antimicrobial peptides onto cellulose nanopaper—Gonzalez et al, 2017). WO2016156878 provides an alternative approach to linking peptides or proteins to cellulose fibers by using virus particles as a linking intermediary.

There remains a need in the industry for cellulose-containing material, to which peptides and proteins can be attached via simple and fast methods, while maintaining a strong and efficacious bond between the cellulose and the peptides or proteins. Specifically, there remains a need for improved antimicrobial material comprising cellulose and antimicrobial peptides or proteins.

The persistence and continued growth of microorganisms can cause several problems, especially when they adhere to each other and/or to living or non-living surfaces in large numbers. In industry, a concentrated population of microorganisms can disrupt normal processes by blocking conduits, pipelines and filters and cause contamination of products. In general use and in the healthcare industry, the microorganisms can become a source of infection and disease.

A compounding problem exists when the microorganisms produce a biofilm, which is a composition comprising one or more different species of microorganisms, such as for example bacteria, archaea, fungi, protozoa, algae and viruses, entrenched within an extracellular matrix comprising polymers, polysaccharides, proteins, nucleic acids and/or lipids. Biofilms form a defensive barrier against commonly used anti-microbial agents offering increased resistance against for example detergents and antibiotics. It is known that this increased resistance can promote recalcitrant infections or antibiotic resistance. Moreover, biofilms are difficult to remove.

In order to prevent or reduce the growth of microorganisms on surfaces and the formation of biofilms, several strategies have been developed. One of these is the use of antimicrobial peptides or proteins (AMP), that are known to be very effective in preventing or destroying biofilms and generally immune to development of bacterial resistance. AMPs are widely used in nature by various organisms as defense against pathogens. Natural occurring AMPs range in size from several to more than 100 amino acids. They vary in structure, but generally share an overall positive charge and a high proportion of hydrophobic residues. This structure allows AMPs to have a broad antipathogenic activity and to selectively associate with highly negatively charged microbial membranes and cause defects sufficient for cell death. AMPs have been used to control the formation of biofilms and to destroy existing biofilms, see for instance Yasir M, Willcox M D P, Dutta D. Action of Antimicrobial Peptides against Bacterial Biofilms. Materials (Basel). 2018 Dec. 5; 11(12):2468. It is also known that some AMPs may retain their activity upon immobilization on solid surfaces. In order to prevent the attachment and growth of microorganisms on cellulose containing material and potential formation of biofilms, it would be therefore be advantageous to have AMPs attached to the cellulose.

Various approaches for linking AMPs to cellulose are described in ACS Appl. Bio Mater. 2020, 3, 8, 4895-4901, but these require multiple chemical processing steps of the cellulose and/or the peptides to obtain modified cellulose and/or peptides suitable for covalent linking.

Green Fluorescent Protein (GFP) was engineered to be fused to a SpyCatcher motif, resulting in GFP-SpyCatcher and recombinantly produced in E. coli. The SpyCatcher motif is purported to readily form isopeptide bonds with its interacting SpyTag partner. This can be used to fuse proteins of interest, which are in turn fused to SpyTag, to GFP-SpyCatcher. When GFP-SpyCatcher is attached to the cellulose particulate material according to the invention this then can be used to attach SpyTag-fused proteins to the GFP-SpyCatcher and thus to the cellulose particulate material. Accordingly, this is a method to attach peptides or proteins to the cellulose particulate material that do not (easily) attach by themselves according to the method of the invention or that are only functional under non-carbonate conditions.

The inventors provide herein also an improved, simple and cost-effective method for the attachment of peptides or proteins to cellulosic absorbent particulate material resulting in an improved composition material comprising cellulose particulate material and peptides or proteins, wherein the peptides or proteins remain functional and strongly attached. ‘Attachment’ of peptides or proteins according to the invention herein is understood to mean the stable colocalization of peptides or proteins with cellulose particulate material after several washing steps with water at room temperature.

Moreover, the inventors provide herein an improved antimicrobial material to address the shortcomings of the prior art by providing a composition material, wherein cellulose particulate material is antimicrobially functionalized via a strong and efficacious attachment of a multitude of antimicrobial peptides or proteins, and optionally a small amount of metal material. The combined antimicrobial effect of the components was found to be more effective than any of the components by themselves, in particular with regard to the prevention of biofilm formation on or adherence of microorganisms to the cellulose. The composition material can be obtained by simple and cost-effective methods.

### Adsorbents for Wound Dressings

In connection with the care and treatment of wounds, the term “wound” is meant to include burns, pressure sores, punctures, ulcers and the like. For a long time, one critical aspect of wound care has been the consideration of the requirements of the epithelium, i. e., that area of new cell growth directly peripheral to the wound which is formed during the healing process, so that healing is facilitated.

Since it has been recognized that healing of the wound occurs in one sense as the epithelium migrates by growth from the periphery inward, care has been taken not to damage unnecessarily or to irritate this new area of growth or existing, compromised periwound tissue. With many dressings, problems can occur during dressing changes. This is particularly true where the dressing adheres to the epithelium or where granulation tissue and new cell growth become intertwined within the matrix of a dressing. In these instances, there is a risk that removal of the dressing will damage the sensitive tissue and new growth on the periphery of the wound thereby causing a regression in the progress of wound healing.

Still, another important consideration in wound care is the needs of the surrounding unwounded skin. The unwounded skin beyond the epithelium is usually in contact with some portion of the wound dressing system which maintains the dressing positioned on the wound. For example, the surrounding skin may be covered for extended periods with a wrap and/or adhesive to hold the dressing in place. Many such dressings can irritate this surrounding skin and compound problems to the patient. This is especially true in the area of leg ulcers wherein the surrounding skin can easily become sensitized by strong medicaments and is often plagued with flaking, scaling and eczema.

It is apparent that, considering the various types of wounds, numerous dressings that are available, and the various stages of healing, there is still a tremendous need for a dressing that functions better than the current dressings, especially with respect to preventing damage to surrounding skin, tissue and new cell growth. In particular, a wound dressing system which protects the epithelium and surrounding non-wounded skin, which wicks away moisture from the wound area, and which does not purposely adhere to the wound or the surrounding area would be a useful addition to the wound care art. A dressing for patients with fragile skin surrounding a wound would be especially beneficial.

An ideal adsorbent for wound dressing should not only absorb exudate but also possess antimicrobial or antibacterial properties. As used herein, “antibacterial” refers to as having an adverse effect on bacteria, particularly disease-causing bacteria. As used herein, the term “antiviral” refers to as having an adverse effect on virus or the spread of viral diseases.

The process of the present invention is now described in further detail. Optionally, the process of the present invention can be carried out as a continuous process, rather than being conducted batch-wise. This has significant advantages in terms of the efficiency of the process. The low viscosity of the mixture formed in the present invention enables continuous processing to be conducted without difficulty.

As used herein, “a,” “an,” “the,” “at least one,” and “one or more” are used interchangeably. Also herein, the recitations of numerical ranges by endpoints include all numbers subsumed within that range. The terms “comprises” and variations thereof do not have a limiting meaning where these terms appear in the description and claims. The words “preferred” and “preferably”, advantageous refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the term “antimicrobial” herein relates to having an adverse effect on a range of pathogenic microorganisms, including bacteria and at least some fungi and viruses. An antimicrobial adsorbent is generally preferred over an antibacterial adsorbent.

As used herein, “antibacterial” refers to as having an adverse effect on bacteria, particularly disease-causing bacteria; and the term “antiviral” refers to as having an adverse effect on spread of viral diseases.

As used herein, the term “nanoparticle” refers to a particle of matter that is between 1-250 nm in diameter as determined by dynamic light scattering.

As used herein, the term “bound” refers to a material physiosorbed (physically absorbed) or chemisorbed (chemically bound) to another material.

As used herein, the term “metallised” refers to the materials wherein metal nanoparticles have been formed.

As used here, the term “attachment” is understood to mean the stable colocalization of peptides or proteins with cellulose particulate material after several washing steps with water at room temperature.

Plant Material: The starting material for the materials according to the present invention comprises herbaceous plant material. The term “herbaceous” as defined herein refers to plants which are annual, biennial or perennial vascular plants. In annual, biennial or perennial vascular plants, the stem matter dies after each season of growth when the plant becomes dormant, i.e. biennial or perennial plants, or dies, i.e. annual plants. Biennial or perennial plants survive unfavorable conditions underground and will regrow in more favorable conditions from such underground portions of the plant, typically stem, roots, or storage organs such as tubers. In contrast, the stems of woody species remain during any period of dormancy, and in a period of further growth will form growth rings which expand the girth of existing tissue. Herbaceous plants are characterized by parenchymal tissue having an abundance of primary cell walls within the tissue. One skilled in the art would also be aware that the mosses and macro algae also consist of an abundance of primary cell walls, and hence are included within the term “herbaceous plant material” as used herein. Herbaceous plant material is preferably used as a starting material within the present invention. Optionally, the starting material of the present invention substantially consists of herbaceous plant material. It can be advantageous for the starting material of the present invention to consist of herbaceous plant material, and thereby exclude wood or wood products. Depending upon the intended end use of the cellulose-containing material, however, it may not, however, be necessary to totally avoid inclusion of non-herbaceous plant material such as wood within the plant starting material.

In particular, the plant material used in the process of the present invention can conveniently include vegetables, for example root vegetables, and fruit. Non-limiting examples of suitable root vegetables include carrot, sugar beet, also commonly referenced as “beet”, turnip, parsnip and swede. Exemplary fruit materials which can be used within the present invention includes apples, pears, citrus and grapes. Optionally, the plant material may be from tubers, for example potato; sweet potato, yam, rutabaga and yucca root can also be used, as well as micro and macro algae.

Generally, it is anticipated that the process of the invention will be operated using waste or coproducts from the plant material after a main product has been extracted, for example sugar beet pellets, vegetable peelings or citrus waste after juicing, jam-making or the like. However this is not strictly necessary, and the process could be operated using vegetable or fruit grown specifically for that purpose. It is also not necessary for the plant material to be used as a starting material in the process of the present invention to comprise material from only one specific plant source.

Optionally, a mixture of materials from different plant sources can be used. For example, the starting material can comprise a mixture of different root vegetables, a mixture of different fruits, a combination of fruit and vegetable(s), including a mixture of root vegetables together with a mixture of fruits.

Generally, the plant material to be used as a starting material for the present invention will not comprise a significant quantity of lignin. Optionally, the starting material for the present invention will comprise less than about 20 wt. % lignin, for example less than about 10 wt. % lignin, for example less than about 5 wt. % lignin, for example less than 2 wt. % lignin, for example less than about 1 wt. % lignin.

A number of methods for the measurement of lignin content are known in the art and include methods such as the “Klason method”, the acetyl bromide method and the thioglycolic acid method. Hatfield and Fukushima (Crop Sci. 45:832-839, 2005) discuss methods of lignin measurement.

The plant material preferably comprises chemically untreated raw plant material, i.e. uncooked. Alternatively, it may have been subjected to an extraction step to remove water soluble compounds, reducing or eliminating the need for an additional washing treatment.

A particularly preferred plant material comprises sugar beet (Beta vulgaris) materials obtained after the sugar juice extraction step. Other suitable materials may be passed through a similar process, e.g. orange peels or apple residue obtained from pressing of juice. Ideally, in this process, the raw plant materials are washed to remove any non-plant material debris or contaminants and leaves. Then typically juice is obtained from those plant materials, by washing and cut up into chips having a thickness in the range of from 0.2 to 0.5 cm. In case of sugar beets, sugar is extracted from these chips typically by contacting the chips with hot extraction water, usually in a counter-current direction in an extraction tower. The crude extract is then usually filtered off, and further worked up. The remaining chips were found to form a particularly good starting material for the present process. In the production of sugar, sugar beets are harvested, washed and processed in sugar beet cutting machines to form chips. The beet chips are subsequently extracted with hot water, at a temperature ranging of from 65° C. to 75° C., generally in a counter-current flow direction, and primarily using a diffusion process, and eventually a physical separation, such as pressing and/or centrifugation. This results in extracted sugar beet chips and sugar-containing raw sugar beet juice.

These extracted sugar beet chips primarily comprise of the cell wall and fibre constituents of the extracted sugar beet. In a subsequent processing stage, the beet chips are typically further dewatered by pressing them in so-called pulp presses, which results in pressed chips and released press water, optionally also using pressing aids. These dewatered and pressed chips are then typically subjected to a thermal removal of the residual water. Herein, the pressed chips are dried at an elevated temperature in rotating and heated drying drums, evaporating residual water and constituents volatile at the conditions. Conventional drying systems apply a so-called high-temperature drying, whereas alternative drying methods make use of indirect drying by means of superheated steam using a fluidized-bed method. Sugar-containing molasses are typically added at this stage if the pressed chips are to be employed as animal feed component. The pressed and dried chips are then usually pelletized, by simultaneously pressing the chips to obtain a compressed composition, and by passing the compressed composition through a granulator such as an extruder or hammer mill, wherein the composition is pelletized. The thus obtained pellets are usually added to animal feedstuff, typically those enriched with sugar-containing molasses.

The plant material may also be treated prior to, or after comminuting to a smaller particle size. Alternatively, the obtained microparticulate matter may be treated. Accordingly, the material may be subjected to a process involving contacting the plant material or obtained with a suitable reagent, such as an alkaline reagent, such caustic soda or lye, and/or water, or an aqueous solution of a peroxide, such as hydrogen peroxide, and/or an oxidative treatment, such as e.g. a hypochlorite.

It is not essential for the reagent to be added simultaneously with the water. However, it is often convenient to add the water and reagent simultaneously. For example, it is possible to premix the reagent with the water and then to add the water-reagent mixture to the plant material, or microparticles. Alternatively, it is possible to add water to the particles of plant material to form an aqueous slurry, and then to add the reagent to the slurry. Advantageously, addition of the water and/or reagent is accompanied by stirring of the resultant mixture to facilitate formation of a homogenous composition. The volume of water to be added is not particularly critical, but may typically be from 2 litres to 30 litres water per kg plant material particles. This is in addition to any solution of reagent which may additionally be added. One of the benefits of the present invention is the relatively high percentage of solids which can be present within the mixture after the addition of water and reagent. In some embodiments, the mixture formed in step (a) can contain more than 2 wt. % solids. In some embodiments, the mixture formed in step (a) can contain at least 3 wt. % solids, for example at least 4 wt. % solids, at least 5 wt. % solids, at least 6 wt. % solids, at least 7 wt. %, at least 8 wt. % solids, at least 9 wt. % solids, or at least 10 wt. % solids.

This treatment step is intended to essentially to not break down the particles, but to remove components that may dissolve easily, and hence later may lead to leaching out of the antimicrobial agents. The process may then be followed by a filtration and washing step to remove unused reagent and soluble components, and drying step.

The cellulosic product, whether washed, treated and washed or directly obtained from a process to remove juices or other desired components is then subjected to a comminution step, e.g. by milling the materials, to obtain a microparticulate material.

The microparticulate material thus obtained was found to be able to super-adsorb fluids, e.g. water in an amount of from 3 to 6 times its dry weight. Also, it was found that the material is doing so very swiftly. Without wishing to be bound to any particular theory, this is believed due to the inherent capillary porosity of the material, which allows wicking of a fluid.

Surprisingly, it was found that the superabsorbent composition had a water absorption capacity (WAC) in the range of from 2 to 10, preferably of from 3.5 to 6.5.

It was found that surprisingly, the materials as such inherently have good antiviral activity against certain viruses, particularly enveloped viruses, such as COVID and influenza viruses. Enveloped viruses preferably have an envelope comprising a lipid bilayer.

This is in particular relevant since it has proven difficult to produce an effective disinfectant that does not readily wash out of the material, in particular when subjected to an industrial or household detergents and washing and drying processes. Hence “wash-out”, or leaching usually occurs, which reduces effectiveness and may cause irritation, or infections. This is a general problem where absorptive packings are placed in contact with significant possibility for dangerous pathogen load, e.g. viral load, or bacterial growth.

In addition, due to the globalization of transport, the emergence of new diseases and pandemics, the uses for a technology that imparts a essentially non-leaching re-useable antimicrobial modification to a variety of materials is duly recognized. Specific areas of use in are described below herein.

And last but not least, sustainable materials would be desired which may simply be subjected to compost preparations, preferably after extraction of valuable antimicrobial components, metals and the like.

### Production of the Superabsorbent Materials

The absorbent particles can be formed using any suitable means. Preferably, water or other liquid is not added to the plant material prior to comminution to form the particles. Thus, the plant material is not in the form of a slurry or suspension during the comminution step. Thus the process can include a step of comminuting plant material in the absence of liquid to form particles of plant material. Optionally, the plant material contains less than 30 wt. % water prior to comminution, for example contains less than 20 wt. % water, for example contains less than 15 wt. % water. In some embodiments, the plant material can be dried (e.g. at ambient temperature or at higher temperatures) before being formed into particles. The comminuted material can be screened to select particles of the desired size.

The particles of plant material can be formed by grinding or milling. For example, the plant material can be processed in a mill or using a grinding apparatus such as a classifier mill to provide particles of the required diameter size.

Preferably, a combination of a mechanically acting mill, i.e. one where the plant materials is crushed and torn apart and thus comminuted between actors, and a subsequent particle sizing is employed, e.g. by gravity or density, or sieving. However, the apparatus used to produce the particles from the plant material is not particularly critical to the successful operation of the process.

Methods for comminuting are not limited in particular, and include, for example, methods by a ball mill, a rod mill, a hammer mill, an impeller mill, a high-speed mixer, attritor mills and/or a disk mill. Of these, preferred are attritor or cell mills, as described for instance in publication WO2013/167851, or in U.S. Pat. Nos. 3,131,875, 3,339,896, 3,084,876, and 3,670,970. In an attritor mill, a high shear field for is attained causing attrition or size reduction of the solid particulate matter. A particularly useful cell mill, coupled with sieves, may be obtained from Atritor Limited, Coventry.

Particle Size and Particle Size Distribution: The particles of plant material used within the process of the present invention have a mean average diameter of from 10 μm to 1000 μm, preferably of from 100 μm to 300 μm. The term “diameter” refers to the measurement across the particle from one side to the other side. One skilled in the art would recognise the particles would not be perfectly spherical, but may be near-spherical, ellipsoid, disc-shaped, or even of irregular shape. One skilled in the art would also be aware that a range of diameters would be present within the starting material. To obtain the benefits of the present invention, it is not necessary to meticulously exclude very small quantities of particles which fall outside the stated particle diameter size. However, inclusion of particles of different diameter sizes within the starting material can, in some circumstances, adversely affect the quality of the end product.

Optionally, at least 60% by volume of the particles have a diameter of from 10 μm to 1000 μm, for example at least 70% by volume of the particles have a diameter of from 10 μm to 1000 μm, or at least 80% by volume of the particles have a diameter of from 10 μm to 1000 μm, or at least 85% by volume of the particles have a diameter of from 10 μm to 1000 μm, or at least 90% by volume of the particles have a diameter of from 10 μm to 1000 μm, or at least 95% by volume of the particles have a diameter of from 10 μm to 1000 μm, or even at least 98% by volume of the particles have a diameter of from 10 μm to 1000 μm. Conveniently 99% by volume of the particles have a diameter of from 10 μm to 1000 μm. In some circumstances it may be advantageous to ensure that substantially all of the particles have a diameter of from 10 μm to 1000 μm.

Depending upon the source of the starting material and/or the intended end use of the cellulose-containing material, it can be advantageous to select particles having a mean average particle diameter size within a narrower range. For example, particles of plant material used within step (a) of the process of the present invention can have a mean average diameter of from 50 μm to 800 μm, or from 100 μm to 600 μm. In some circumstances, at least 60% by volume of the particles have a diameter of from 50 μm to 800 μm, for example at least 70% by volume of the particles have a diameter of from 50 μm to 800 μm, or at least 80% by volume of the particles have a diameter of from 50 μm to 800 μm, or at least 85% by volume of the particles have a diameter of from 50 μm to 800 μm, or at least 90% by volume of the particles have a diameter of from 50 μm to 800 μm, or at least 95% by volume of the particles have a diameter of from 50 μm to 800 μm, or even at least 98% by volume of the particles have a diameter of from 50 μm to 800 μm. Conveniently, 99% by volume of the particles have a diameter of from 50 μm to 800 μm. In some circumstances it may be advantageous to ensure that substantially all of the particles have a diameter size of from 50 μm to 800 μm.

In a preferable embodiment of the disclosure, the particles of plant material used within step (a) of the process of the present invention can have a mean average diameter of from 200 μm to 400 μm. In some circumstances, at least 60% by volume of the particles have a diameter of from 200 μm to 400 μm, for example at least 70% by volume of the particles have a diameter of from 200 μm to 400 μm, or at least 80% by volume of the particles have a diameter of from 200 μm to 400 μm, or at least 85% by volume of the particles have a diameter of from 200 μm to 400 μm, or at least 90% by volume of the particles have a diameter of from 200 μm to 400 μm, or at least 95% by volume of the particles have a diameter of from 200 μm to 400 μm, or even at least 98% by volume of the particles have a diameter of from 200 μm to 400 μm. Conveniently 99% by volume of the particles have a diameter of from 200 μm to 400 μm. In some circumstances it may be advantageous to ensure that substantially all of the particles have a diameter of from 200 μm to 400 μm.

In an alternative, but equally preferable, embodiment of the disclosure in which the process for preparing an antimicrobial cellulose-containing superabsorbent composition from an herbaceous plant material comprises the step of at least once contacting the microparticles or microparticle film with an antimicrobial agent precursor under conditions inductive of the formation, attachment or binding of an antimicrobial agent, the particles of plant material used within step (a) of the process of the present invention can have a mean average diameter of from 75 μm to 400 μm. In some circumstances, at least 60% by volume of the particles have a diameter of from 100 μm to 300 μm, for example at least 70% by volume of the particles have a diameter of from 100 μm to 300 μm, or at least 80% by volume of the particles have a diameter of from 100 μm to 300 μm, or at least 85% by volume of the particles have a diameter of from 100 μm to 300 μm, or at least 90% by volume of the particles have a diameter of from 200 μm to 400 μm, or at least 95% by volume of the particles have a diameter of from 200 μm to 400 μm, or even at least 98% by volume of the particles have a diameter of from 200 μm to 400 μm. Conveniently 99% by volume of the particles have a diameter of from 200 μm to 400 μm. In some circumstances it may be advantageous to ensure that substantially all of the particles have a diameter of from 200 μm to 400 μm.

Particles of the required diameter and within the predetermined particle size distribution can be selected using known methods, including (but not limited to) sieving the particle mixture with one or more sieves of known sieve size.

For example, passing the material sample through a sieve having a mesh size of 500 μm will only allow particles having a particle diameter of 500 μm of less to pass through. The sieved material can then be sieved again using a sieve having a smaller mesh size, for example a mesh size of 300 μm. The particles retained on the smaller mesh (i.e. which do not pass through) will have a particle size distribution of 200 μm and range in size from 300 μm to 500 μm. Of course, sieves of alternative sieve size and in different combinations can be used to obtain any required particles diameter size range and particle size distribution. Alternatively, a classifier mill or other suitable means can be used to select particles of the required particle size and size distribution.

Optional step (c) may comprise washing, or, if desired, neutralising the hydrated mixture to form a washed hydrated mixture.

As indicated above, step (c) can include one or more washing steps. Typically, washing requires the cellulose material to be separated from the liquid fraction, and then re-suspended (optionally with agitation or stirring) in clean liquid, such as water. The washing step essentially removes any excess reagent, and also any soluble by-products formed in step (a).

Also, if a treatment has been applied, the mixture may be washed, and/or neutralised, to a desired pH. Neutralising the mixture of step (b) after the end point pH has been reached can reduced or even eliminate the requirement for a washing step, thereby reducing the amount of water consumed during the manufacturing process, which is an important environment consideration. Neutralisation can be achieved by addition of an appropriate amount of an acid in an amount sufficient to change the pH of the mixture to a neutral pH. The acid can be added in any convenient form, but typically will be added as a powder or in the form of an aqueous solution. Alkalis such as sodium hydroxide, potassium hydroxide, calcium carbonate or the like can conveniently be used for the treatment. Optionally, once the hydrated mixture has been neutralized (and optionally mixed therewith}, the cellulose-containing material can be separated from the liquid fraction by any suitable means.

Alternatively, the step of neutralisation can be performed after the cellulose-containing material has been separated from the liquid fraction. For example, the cellulose-containing material can be separated and then re-suspended before a suitable amount of acid is added. Alternatively, the separated cellulose-containing material can simply be suspended in an acidic solution. The step of separating the cellulose-containing material from the liquid fraction can be achieved using any suitable apparatus or process, including without limitation filtration (simple or vacuum filtration), centrifugation, membrane filtration etc. A woven filter can be used. Alternatively a mesh filter can be used. Optionally, where filtration is used during the washing step, the filter has a pore size of 200 μm or less, for example has a pore size of 100 μm to 200 μm. A smaller pore size can also be used.

Optionally, the washing step (c), and/or neutralising step (c), if present, is conducted in a manner which is compatible with a continuous manufacturing process. For example a filter at an angle of approximately 450 to the horizontal may advantageously be used, with the material to be filtered being dropped onto the filter from above so that liquid drains through the filter whilst solids are retained on the upper surface of the filter. The angle of the filter cause these retained solids to slide gently down the filter's upper surface onto a belt, or into a hopper or other receptacle ready for further processing. Alternatively a belt filter press can be used.

Optional step (d): Once step (c) is complete (including any optional washing and/or neutralising steps), the obtained material may be isolated, and water removed. The material may be dried to touch dryness; e.g. comprising a water content equivalent to exposure of dry material to average air humidity; or dried further and package under exclusion of air humidity. Methods for drying are well-know, and include drying cylinders, rotating drums, belts and the like, typically heated by superheated steam or hot air; and also may include reduced pressure (vacuum drying). Preferred is heat drying under reduced pressure, i.e. heated vacuum drying. Drying may preferably be done by subjecting the materials to air flow at elevated temperatures in rotating and heated drying drums, evaporating residual water and constituents volatile at the conditions. Conventional drying systems apply a so-called high-temperature drying, whereas alternative drying methods make use of indirect drying by means of superheated steam using a fluidized-bed method.

Additionally, the materials, whether obtained in step (d) or (e), may be modified by adding functional materials, e.g. additional antimicrobial compounds; colouring or pigmentation, or any other useful modifications, such as shaping or compressing into certain shapes or products, optionally with packaging.

Optional step e: After step (c) or optionally after step (d), the obtained the microparticles are contacted with an antimicrobial agent precursor under conditions inductive of the formation, attachment or binding of an antimicrobial agent.

In one embodiment, the cellulose microparticles are functionalised with antimicrobial nanoparticles so as to form an antimicrobial agent.

The antimicrobial nanoparticles may be selected from any nanoparticular material wherein 95 wt. % of the particles have a mean average diameter of 1-250 nm. The antimicrobial nanoparticles are bound to the surface of the fibres of the microparticles. It has been surprisingly found that nanoparticles bound to the cellulose active material do not de-attach from the cellulose active material under conditions emulating washing, even at 70° C. Without wishing to be bound by theory, it is believed that the nanoparticles physical incorporation within the fibrous network of the cellulose active material particles provides additional protection from physical ablation of the nanoparticles during washing.

Preferably, the antimicrobial nanoparticles are selected from silver nanoparticles (AgNPs), gold nanoparticles [AuNPs] or copper nanoparticles (CuNPs). More preferably, the antimicrobial nanoparticles are selected from silver nanoparticles (AgNPs), or copper nanoparticles (CuNPs). Most preferably, the antimicrobial nanoparticles are selected from silver nanoparticles (AgNPs).

Optionally, the antimicrobial nanoparticles may be pre-formed and contacted with the microparticles to bind the antimicrobial nanoparticles to the microparticles so as to form an antimicrobial agent. The preformed antimicrobial nanoparticles therefore as an antimicrobial agent precursor and by binding to the fibres of the microparticles form an antimicrobial agent. This can be achieved by immersing the microparticles into a colloidal suspension bearing the preformed nanoparticles, followed by isolation of the nanoparticle-binding microparticles by filtration and washing of said isolated nanoparticle-binding microparticles. Preferably, the nanoparticles are provided as an aqueous solution.

Preferably, the pre-formed antimicrobial nanoparticles are selected from silver nanoparticles (AgNPs), gold nanoparticles [AuNPs] or copper nanoparticles (CuNPs). More preferably, the pre-formed antimicrobial nanoparticles are selected from silver nanoparticles (AgNPs) or copper nanoparticles (CuNPs). Most preferably, the pre-formed antimicrobial nanoparticles are selected from silver nanoparticles (AgNPs).

Alternatively, the antimicrobial nanoparticles may be formed in-situ. Here the nanoparticle pre-cursors act as an antimicrobial agent precursor and by forming nanoparticles at the surface of the fibres of the microparticles form an antimicrobial agent. Suitable nanoparticle pre-cursors may be selected from known nanoparticle precursors, such as copper salts, silver salts or gold salts. Particularly favoured nanoparticle precursors are copper sulfate (CuSO₄), copper acetate (Cu(OAc)₂), silver nitrate (AgNO₃) and the chlorides of gold, including gold (Ill) chloride (Au₂Cl₆), chloroauric acid (HAuCl₄) and gold (i) chloride (AuCl), preferably copper sulfate (CuSO₄), silver nitrate (AgNO₃) and chloroauric acid (HAuCl₄), most preferably silver nitrate (AgNO₃).

The nanoparticle precursors may be provided as a solution, preferably as an aqueous solution.

Preferably, the nanoparticle precursors are provided as a 0.01-50 μmicromolar (mMol dm⁻³) solution on the basis of the metal containing compound, more preferably 0.1-20 μmicromolar, most preferably 1-10 micromolar.

Surprisingly, the inventors found that no reducing agent was required to form nanoparticles in situ when contacted with the microparticles. Optionally, additional reducing agents may be used to expedite nanoparticle formation.

The solution of nanoparticle precursors may be contacted with the microparticles by immersion of the microparticles in a solution of the nanoparticle precursors. Alternatively, a solution of nanoparticle precursors may be spray coated onto the microparticles.

Preferably, nanoparticle formation is performed under illumination with light, preferably light of a wavelength of 300-500 nm, more preferably 350-450 nm, yet more preferably 380-420 nm, most preferably 390-400 nm.

Preferably, nanoparticle formation is conducted at from 0 to 150° C., more preferably from 10 to 110° C., yet more preferably from 15 to 60° C. and most preferably from 20 to 30° C.

Preferably, nanoparticle formation is conducted as part of a continuous process, wherein the microparticles prepared by steps described above are contacted with at least one. For example, the microparticles prepared by steps described above may be spray coated with a solution of nanoparticle precursors. Suitable nanoparticle pre-cursors may be selected from known nanoparticle precursors, such as copper salts, silver salts or gold salts. Particularly favoured nanoparticle precursors are copper sulfate (CuSO₄), copper acetate (Cu(OAc)₂), silver nitrate (AgNO₃) and the chlorides of gold, including gold (Ill) chloride (Au₂Cl₆), chloroauric acid (HAuCl₄) and gold (i) chloride (AuCl), preferably copper sulfate (CuSO₄), silver nitrate (AgNO₃) and chloroauric acid (HAuCl₄), most preferably silver nitrate (AgNO₃). The particularly favoured nanoparticles may be provided as an aqueous solution. More preferably, nanoparticle formation is conducted as part of a continuous process, wherein the microparticles prepared by steps described above are contacted with at least one nanoparticle precursor followed by irradiation with light of a wavelength of 300-500 nm. Suitable nanoparticle pre-cursors may be selected from known nanoparticle precursors, such as copper salts, silver salts or gold salts. Particularly favoured nanoparticle precursors are copper sulfate (CuSO₄), copper acetate (Cu(OAc)₂), silver nitrate (AgNO₃) and the chlorides of gold, including gold (Ill) chloride (Au₂Cl₆), chloroauric acid (HAuCl₄) and gold (i) chloride (AuCl), preferably copper sulfate (CuSO₄), silver nitrate (AgNO₃) and chloroauric acid (HAuCl₄), most preferably silver nitrate (AgNO₃). The particularly favoured nanoparticles may be provided as an aqueous solution. nanoparticle formation is performed under illumination with light, preferably light of a wavelength of 300-500 nm, more preferably 350-450 nm, yet more preferably 380-420 nm, most preferably 390-400 nm.

Most preferably, nanoparticle formation is conducted as part of a continuous process, wherein the microparticles prepared by steps described above are contacted with an aqueous solution of silver nitrate (AgNO₃), followed by irradiation with of a wavelength of 300-500 nm.

In an alternative embodiment, the cellulose active material particles are functionalised with antimicrobial peptide and/or proteins.

Preferably, the peptides and/or proteins are antimicrobial. More preferably the antimicrobial peptides or proteins comprise peptides or proteins having an amino acid sequence selected from the group consisting of AU1, AU2, AU3, 1037, LF1-11, KR12, lactoferrampin, FK-16 and Dispersin B (SEQ ID NOs 1 to 9). AU1 has an amino acid sequence of SEKLFFGASL (SEQ ID NO 1). AU2 has an amino acid sequence of SEKLWWGASL (SEQ ID NO 2). AU3 has an amino acid sequence of GASLWWSEKL (SEQ ID NO 3). Lactoferrampin has an amino acid sequence of WNLLRQAQEKFGKDKSP (SEQ ID NO 7). FK-16 has an amino acid sequence of FKRIVQRIKDFLRNLV (SEQ ID NO 8) or FKRIVQRIKDFLRNLV-amide (SEQ ID NO: 10). 1037 has an amino acid sequence of KRFRIRVRV (SEQ ID NO 4) or KRFRIRVRV-amine (SEQ ID NO:11). The amino acid sequences and their SEQ ID NOs of the preferred antimicrobial peptides and proteins according to the invention are set out in Table A below.

TABLE A
Amino acid sequences and SEQ ID NOs of the
preferred antimicrobial peptides and proteins
according to the invention

| SEQ | | |
|---|---|---|
| ID | | |
| NO | Name | Sequence |
| 1 | AU1 | SEKLFFGASL |
| 2 | AU2 | SEKLWWGASL |
| 3 | AU3 | GASLWWSEKL |
| 4 | 1037 | KRFRIRVRV |
| 5 | LF1-11 | GRRRSVQWCAV |
| 6 | KR12 | KRIVQRIKDFLR |
| 7 | Lactoferrampin | WNLLRQAQEKFGKDKSP |
| 8 | FK-16 | FKRIVQRIKDFLRNLV |
| 9 | Dispersin B | NCCVKGNSIYPQKTSTKQTGLMLDI |
| | | ARHFYSPEVIKSFIDTISLSGGNFL |
| | | HLHFSDHENYAIESHLLNQRAENAV |
| | | QGKDGIYINPYTGKPFLSYRQLDDI |
| | | KAYAKAKGIELIPELDSPNHMTAIF |
| | | KLVQKDRGVKYLQGLKSRQVDDEID |
| | | ITNADSITFMQSLMSEVIDIFGDTS |
| | | QHFHIGGDEFGYSVESNHEFITYAN |
| | | KLSYFLEKKGLKTRMWNDGLIKNTF |
| | | EQINPNIEITYWSYDGDTQDKNEAA |
| | | ERRDMRVSLPELLAKGFTVLNYNSY |
| | | YLYIVPKASPTFSQDAAFAAKDVIK |
| | | NWDLGVWDGRNTKNRVQNTHEIAGA |
| | | ALSIWGEDAKALKDETIQKNTKSLL |
| | | EAVIHKTNGDE |
| 10 | “FKRIVQRIKDFLRNLV- | FKRIVQRIKDFLRNLV-amide |
| | amide” | |
| 11 | “KRFRIRVRV-amine” | KRFRIR VR V-amine |

Dispersin B is a family 20 β-hexosaminidase originating from the oral pathogen Aggregatibacter actinomycetemcomitans, also known as Actinobacillus actinomycetemcomitans.

Preferably, the peptides and/or proteins have a metal binding domain, more preferably a copper, silver or gold binding domain, most preferably a silver binding domain.

In an alternative embodiment, the cellulose active material particles are functionalised with both (i) antimicrobial nanoparticles and (ii) antimicrobial peptides and/or proteins.

Preferably, the peptides and/or proteins have a metal binding domain, more preferably a copper, silver or gold binding domain, most preferably a silver binding domain.

The antimicrobial nanoparticles may be selected from any nanoparticular material wherein 95 wt. % of the particles have a mean average diameter of 1-250 nm. The antimicrobial nanoparticles are bound to the surface of the fibres of the microparticles. It has been surprisingly found that nanoparticles bound to the cellulose active material do not de-attach from the cellulose active material under conditions emulating washing, even at 70° C. Without wishing to be bound by theory, it is believed that the nanoparticles physical incorporation within the fibrous network of the cellulose active material particles provides additional protection from physical ablation of the nanoparticles during washing.

Preferably, the antimicrobial nanoparticles are selected from silver nanoparticles (AgNPs), gold nanoparticles [AuNPs] or copper nanoparticles (CuNPs). More preferably, the antimicrobial nanoparticles are selected from silver nanoparticles (AgNPs) or copper nanoparticles (CuNPs). Most preferably, the antimicrobial nanoparticles are selected from silver nanoparticles (AgNPs).

Optionally, the antimicrobial nanoparticles may be pre-formed and contacted with the microparticles to bind the antimicrobial nanoparticles to the microparticles so as to form an antimicrobial agent. The preformed antimicrobial nanoparticles therefore as an antimicrobial agent precursor and by binding to the fibres of the microparticles form an antimicrobial agent. This can be achieved by immersing the microparticles into a colloidal suspension bearing the preformed nanoparticles, followed by isolation of the nanoparticle-binding microparticles by filtration and washing of said isolated nanoparticle-binding microparticles. Preferably, the nanoparticles are provided as an aqueous solution.

Preferably, the antimicrobial nanoparticles may be formed in-situ. Here the nanoparticle pre-cursors act as an antimicrobial agent precursor and by forming nanoparticles at the surface of the fibres of the microparticles form an antimicrobial agent. Suitable nanoparticle pre-cursors may be selected from known nanoparticle precursors, such as copper salts, silver salts or gold salts. Particularly favoured nanoparticle precursors are copper sulfate (CuSO₄), copper acetate (Cu(OAc)₂), silver nitrate (AgNO₃) and the chlorides of gold, including gold (Ill) chloride (Au₂Cl₆), chloroauric acid (HAuCl₄) and gold (i) chloride (AuCl), preferably copper sulfate (CuSO₄), silver nitrate (AgNO₃) and chloroauric acid (HAuCl₄), most preferably silver nitrate (AgNO₃).

Forming antimicrobial nanoparticles in-situ confers the advantage of particularly great resistance to the thereby formed nanoparticles “washing-out” or leaching from the material. Without being bound by theory, it is believed that formation of the nanoparticles in-situ may result in the nanoparticles forming within the network of cellulosic fibres of the microparticles, thereby physically trapping the fully formed nanoparticles within the network.

The nanoparticle precursors may be provided as a solution, preferably as an aqueous solution.

Preferably, the nanoparticle precursors are provided as a 0.01-50 μmicromolar (mMol dm⁻³) solution on the basis of the metal containing compound, more preferably 0.1-20 μmicromolar, most preferably 1-10 micromolar.

Surprisingly, the inventors found that no reducing agent was required to form nanoparticles in situ when contacted with the microparticles. Optionally, additional reducing agents may be used to expedite nanoparticle formation.

The solution of nanoparticle precursors may be contacted with the microparticles by immersion of the microparticles in a solution of the nanoparticle precursors. Alternatively, a solution of nanoparticle precursors may be spray coated onto the microparticles.

Preferably, nanoparticle formation is performed under illumination with light, preferably light of a wavelength of 300-500 nm, more preferably 350-450 nm, yet more preferably 380-420 nm, most preferably 390-400 nm.

Preferably, nanoparticle formation is conducted at from 0 to 150° C., more preferably from 10 to 110° C., yet more preferably from 15 to 60° C. and most preferably from 20 to 30° C.

In a particularly favoured combination, the peptides and/or proteins have a copper, silver or gold binding domain and the nanoparticles are formed in-situ from corresponding a copper, silver or gold containing nanoparticle precursor, of which the combination of peptides and/or proteins having a silver binding domain and a silver containing nanoparticle precursor is most favoured. Without being bound by theory, it is believed that the metal binding domain of the peptide and/or protein and the nanoparticle act to enhance “attachment” of the peptides and/or proteins to the cellulose-containing microporous superabsorbent composition. The cellulose-containing microporous superabsorbent composition comprising both (i) antibiofilm/antibacterial peptides/proteins and (ii) antimicrobial nanoparticles surprisingly demonstrated synergistic antibacterial properties.

Step f: After step (e) the antimicrobially modified microporous superabsorbent composition is isolated. This can be achieved by isolation of the antimicrobially modified microporous superabsorbent composition by filtration, followed by washing.

Optional step g: a film may be formed from the antimicrobially modified microporous superabsorbent composition.

In preferable embodiment, the process for preparing an antimicrobial and cellulose-containing microporous superabsorbent composition comprises:
(1) performing the process for preparing a preparing an antimicrobial nanoparticle-containing cellulose-containing microporous superabsorbent composition according to any of the suitable embodiments describes above;
(2) performing the process for preparing a preparing an antimicrobial protein-containing and/or peptide-containing cellulose-containing microporous superabsorbent composition according to any of the suitable embodiments describes above; and
(3) blending the antimicrobial nanoparticle-containing cellulose-containing microporous superabsorbent composition and the antimicrobial protein-containing and/or peptide-containing cellulose-containing microporous superabsorbent composition.

This embodiment advantageously allows for a simplified preparation of a superabsorbent compositions comprising both anti-microbial nanoparticles and antimicrobial proteins and/or peptides.

In a particularly preferable embodiment E1, the present disclosure relates to a process for preparing a cellulose-containing microporous superabsorbent composition from a herbaceous plant material, the process comprising the step of:
a) comminuting dry granulated herbaceous plant material to form microparticles having an average particle diameter of from 100 μm to 800 μm;
optionally, b) contacting the microparticles with an aqueous solution; optionally comprising an alkaline reagent; neutralising the mixture, and/or washing the aqueous solution; and removing at least part of the fluids from the mixture, and optionally, drying the obtained material;
to obtain the cellulose-containing microporous superabsorbent composition.

The present disclosure further relates to embodiment E2, which is according to embodiment E1, wherein the starting material comprises less than 20 wt. % lignin.

The present disclosure further relates to embodiment E3, which is according to embodiment E1 or E2, wherein the superabsorbent composition has a water absorption capacity (WAC) in the range of from 2 to 10.

The present disclosure further relates to embodiment E4, which is according to any of embodiments E1-3, wherein the plant material is selected from root vegetables including carrot, sugar beet, turnip, parsnip and swede; fruit materials including apples, pears, citrus and grapes; and/or tubers, including potato; sweet potato, yam, rutabaga and yucca root; preferably sugar beet.

The present disclosure further relates to embodiment E5, which is according to embodiment E4, wherein the material comprises sugar beet (Beta vulgaris) materials obtained after the sugar juice extraction step; orange peels or apple residue obtained from pressing of juice; and wherein the materials are subjected to washing to remove any non-plant material debris or contaminants and leaves; then pressing of the juice, and washing and cutting up into chips having a thickness in the range of from 0.2 to 0.5 cm; and optionally extracting sugar or volatiles from the chips, by contacting the chips with an extractant.

The present disclosure further relates to embodiment E6, which is according to embodiment E1, further comprising a step (c) of modifying and/or shaping the materials obtained in step (b) or step (a).

The present disclosure further relates to embodiment E7, which is a cellulosic superabsorbent material obtainable by the process according to any of embodiments E1-6 comprising a fluid-superabsorbent volume area able to absorb of from 3 to 6 times of the original weight within 30 seconds, and exhibiting a virucidal activity as expressed by a reduction in viral titre of influenza A and/or human coronavirus of above 90%, as determined pursuant to ISO18184:2019.

The present disclosure further relates to embodiment E8, which is the material according to embodiment E7, for use in absorbing fluids, preferably in absorbing aqueous fluids, menses, bodily fluids, skin, cosmetic compositions, wound exudates, and/or oil spills.

The present disclosure further relates to embodiment E9, which is the material according to embodiment E6, wherein the material is shaped into, or comprised in a wound dressing, a sanitary pad, a tampon, an absorbent dressing, a diaper, a sponge, a sanitary wipe, isolation and surgical gowns, gloves, surgical scrubs, sutures, sterile packaging, floor mats, burn dressings, mattress cover, bedding, soft furnishings, air filters for autos, planes or HVAC systems, military protective garments, face masks, devices for protection against biohazards and biological warfare agents, lumber, paper, cardboard, meat or fish packaging material, apparel for food handling, and other surfaces required to exhibit a non-leaching antimicrobial property and to release over time portions of biologically or chemically active compounds, or as a particulate matter for absorbing spilled fluids.

The present disclosure further relates to embodiment E10, which is a functionalised superabsorbent composition comprising:
i) a material according to embodiment E5; and
ii) an antimicrobial, colouring or otherwise functional agent selected from antibiotics, analgesics, anti-inflammatories, oxidizing agents, metalloproteinase inhibitors, proteins, peptides, and fragrances adhered to the material.
   Uses of the Cellulose-Containing Material: The present materials are superabsorbent with an advantageous liquid storage capacity combined with a liquid wicking efficacy. Hence, these superabsorbent materials are economically viable for use in absorbent articles and are fully biodegradable, so that disposal of the absorbent articles used is environmentally friendly.

The term “absorbent article” generally refers to a device that can absorb and contain fluids. As used herein, absorbent articles include baby sanitary products such as diapers, baby wipes, bowel training pants and other disposable garments; Feminine hygiene products such as sanitary napkins, wipes, sanitary pads, pantiliners, panty shields, tampons and tampon applicators; Adult sanitary products such as wipes, pads, incontinence products, urine shields, furniture pads, bed pads and head bands; Public, industrial and household products such as wipes, covers, filters, paper towels, bath tissues and facial tissues; Nonwovens, such as nonwoven rolls; Home comfort products, such as pillows, pads, cushions and masks; And professional and consumer hygiene products, including but not limited to surgical drapes, hospital gowns, wipes, wraps, covers, bands, filters and disposable garments.

Preferred or alternative features of each aspect or embodiment of the invention apply mutatis mutandis to each aspect or embodiment of the invention (unless the context demands otherwise).

The term “comprising” as used herein means consisting of, consisting essentially of, or including and each use of the word “comprising” or “comprises” can be independently revised by replacement with the term “includes”, “consists essentially of or “consists of”.

Any modifications and/or variations to described embodiments that would be apparent to one of skill in art are hereby encompassed. Whilst the invention has been described herein with reference to certain specific embodiments and examples, it should be understood that the invention is not intended to be unduly limited to these specific embodiments or examples.

The absorbent materials according to the invention, optionally modified, advantageously may be used in wound dressing, sanitary pad, a tampon, an intrinsically antimicrobial absorbent dressing, a diaper, toilet paper, a sponge, a sanitary wipe, food preparation surfaces, gowns, gloves, surgical scrubs, sutures, needles, sterile packings, floor mats, lamp handle covers, burn dressings, gauze rolls, blood transfer tubing or storage container, mattresses, applicators, exam table coves, head covers, cast liners, splint, paddings, lab coats, air filters for autos, planes or HVAC systems, military protective garments, face masks, devices for protection against biohazards and biological warfare agents, food packaging material, and other materials that would profit from biodegradable and antimicrobial properties.

In connection with the care and treatment of wounds, the term “wound” is meant to include burns, pressure sores, punctures, ulcers and the like. For a long time, one critical aspect of wound care has been the consideration of the requirements of the epithelium, i. e., that area of new cell growth directly peripheral to the wound which is formed during the healing process, so that healing is facilitated.

The above summary of the present invention is not intended to describe each disclosed embodiment or every implementation of the present invention. The description that follows more particularly exemplifies illustrative embodiments.

The present invention is now further described with reference to the following non-limiting examples.

### Example 1—Preparation of Cellulose-Containing Microporous Superabsorbent Composition

Sugar-beet pellets obtained from a sugar extraction process were subjected to a milling step in an attritor mill, selecting a composition comprising at a weight average particles size of from 100 μm to 300 μm, and subsequently subjected to a water washing and drying step, to obtain a cellulose-containing microporous superabsorbent composition.

### Example 2—Preparation of antimicrobial cellulose-containing microporous superabsorbent composition comprising silver nanoparticles

70 g of the cellulose-containing microporous superabsorbent composition of Example 1 was treated with 700 μmL of a 5 mM aqueous solution of AgNO₃ at 25° C. for two hours. The suspension was filtered, the retained solids then dried under a flow of air at 25° C. for 12 hours. The dried material was then suspended in 700 μmL of water at 25° C. for two hours. The suspension was filtered, the retained solids then dried under a flow of air at 25° C. The dried material was then washed with water three times. Each water wash consisted of suspending the solid material in 1400 μmL of water for 10 μminutes at 25° C., followed by isolation of the solid by filtration. The isolated, thrice washed solid material was then dried under a flow of air at 25° C. for 12 hours. ICP-MS analysis established 681(±11) mg/kg of silver was incorporated into the material, which means approximately 12% of the silver present in the aqueous silver salt was incorporated into the material on an elemental basis.

### Example 3—Preparation of antimicrobial cellulose-containing microporous superabsorbent composition comprising gold nanoparticles

The cellulose-containing microporous superabsorbent composition of Example 170 g was treated with 700 μm of a 5 mM aqueous solution of HAuCl₄·3H₂O at 25° C. for two hours. The suspension was filtered, the retained solids then dried under a flow of air at 25° C. The dried material was then suspended in 700 μmL of water at 25° C. for two hours. The suspension was filtered, the retained solids then dried under a flow of air at 25° C. The dried material was then washed with water three times. Each water wash consisted of suspending the solid material in 1400 μmL of water for 10 μminutes at 25° C., followed by isolation of the solid by filtration. The isolated, thrice washed solid material was then dried under a flow of air at 25° C. ICP-MS analysis established 7500(±399) mg/kg of silver was incorporated into the material, which means approximately 80% of the fold present in the aqueous sold salt was incorporated into the material on an elemental basis.

TABLE 1
Fluid absorbance of antimicrobial cellulose-containing
microporous superabsorbent compositions

| | | Example 2 | Example 3 |
|---|---|---|---|
| Material | Example 1 | [AgNP] | [AuNP] |
| Water absorbance | 4.957 | 6.145 | 4.967 |
| [g(water)/g(material)] | | | |

### Example 4—Elemental Analysis (ICP-MS) of Materials

Materials prepared according to Examples 2 and 3 were digested in aqua regia (21% HCl/9% HNO₃) and the resultant aqueous solution analysed in triplicate by Inductively Coupled Plasma Mass Spectrometry (ICP-MS) using a Perkin Elmer AVIO 500 in a laboratory accredited under ISO 17025. Samples were diluted if required to bring results within the calibration range. Standard and QC solutions made stocks traceable NIS and the method utilises multi-point calibration with the following standards:10, 6, 4, 2 and 1 ppm metal. The results are reproduced in Table 2.

TABLE 2

| Material | Example 2 | Example 3 |
|---|---|---|
| Silver (mg/kg) | 681(±11) | 0 |
| Gold (mg/kg) | 0 | 7500(±399) |
| % incorporation of available metal | 12 | 80 |
| into material | | |

### Example 5—Antiviral Activity of Antimicrobial Cellulose-Containing Microporous Superabsorbent Compositions According to the Invention

The antiviral activity of antimicrobial cellulose-containing microporous superabsorbent compositions according to the invention were evaluated by the protocol of ISO 18184:2019. Formulations comprising cellulose active material were tested for their viricidal activity against Influenza A virus or Human coronavirus NL63 at a contact time of 2 h relative to a reference control, following ISO18184:2019. The formulations tested were (1) cellulose-containing microporous superabsorbent composition of Example 1, (2) silver metallised (AgNP) cellulose-containing microporous superabsorbent composition of Example 3 and (4) gold metallised (AuNP) cellulose-containing microporous superabsorbent composition of Example 3. The results are shown in Tables 3 and 4.

TABLE 3
Effect against Influenza A. A value of 2.0 > Mv ≥ 1.0 indicates good
antiviral effect. A value of 3.0 > Mv ≥ 2.0 indicates very good antiviral
effect. A value of Mv > 3.0 indicates excellent antiviral effect.

| | | Mv | | Affects | |
|---|---|---|---|---|---|
| cellulose active material form vs Influenza A | % | (antiviral | | cell | ISO |
| [H1N1] | reduction | activity) | Cytotoxic | sensitivity | compliant |
| Cellulose-containing microporous | 99.9 | 3.17 | N | Y | N* |
| superabsorbent composition of Example 1 | | | | | |
| Silver metallised (AgNP) cellulose-containing | 99.9 | 3.93 | N | N | Y |
| microporous superabsorbent composition of | | | | | |
| Example 2 | | | | | |
| Gold metallised (AuNP) cellulose-containing | 99.9 | 3.93 | N | N | Y |
| microporous superabsorbent composition of | | | | | |
| Example 3 | | | | | |

*Affected cell susceptibility not valid for ISO18184.

TABLE 4
Effect against Influenza A. A value of 2.0 > Mv ≥ 1.0 indicates good
antiviral effect. A value of 3.0 > Mv ≥ 2.0 indicates very good antiviral
effect. A value of Mv ≥ 3.0 indicates excellent antiviral effect.

| | | Mv | | Affects | |
|---|---|---|---|---|---|
| cellulose active material form vs Human | % | (antiviral | | cell | ISO |
| Coronavirus [NL63] | reduction | activity) | Cytotoxic | sensitivity | compliant |
| Cellulose-containing microporous | 99.9 | 3.53 | N | N | Y |
| superabsorbent composition of Example 1 | | | | | |
| Silver metallised (AgNP) cellulose-containing | 99.9 | 3.22 | N | N | Y |
| microporous superabsorbent composition of | | | | | |
| Example 2 | | | | | |
| Gold metallised (AuNP) cellulose-containing | 99.9 | 3.25 | N | N | Y |
| microporous superabsorbent composition of | | | | | |
| Example 3 | | | | | |

As can be seen from Tables all the cellulose-containing microporous superabsorbent compositions exhibited good to excellent viricidal activity with a 99.9% reduction in viral titre (both influenza A and human coronavirus) at a contact time of 2 h relative to a reference control, following ISO18184:2019 accredited procedures. Under these conditions all were ISO compliant except cellulose-containing microporous superabsorbent composition of Example 1 since although they had potent antiviral activity, they were found to slightly influence growth of the type of cells used in the influenza assay.

### Example 6—Antibacterial Activity of Antimicrobial Cellulose-Containing Microporous Superabsorbent Compositions According to the Invention

The antibacterial activity of antimicrobial cellulose-containing microporous superabsorbent compositions according to the invention were evaluated. Formulations comprising cellulose active material were tested for their antibacterial control activity against Escherichia coli, Pseudomonas syringae and Saccharomyces cerevisiae cultures. The formulations tested were (1) cellulose-containing microporous superabsorbent composition of Example 1, (2) silver metallised (AgNP) cellulose-containing microporous superabsorbent composition of Example 2 and (3) gold metallised (AuNP) cellulose-containing microporous superabsorbent composition of Example 3. The protocol used contacted 100 μl aqueous cell suspensions with 100 μmg of antimicrobial cellulose-containing microporous superabsorbent composition for 2 hours at ambient conditions, 1 mL of culture media added, vortexed and plated, and triplicate counts of microbial colonies were obtained. By comparing numbers of colonies obtained from the cell suspensions exposed to the materials with colony numbers obtained from suspensions that had no contact with the materials we were able to calculate the capacity of the materials to inhibit microbial growth (% control). % control data are reproduced in Table 5.

TABLE 5
Control Data for microbial growth (% control).

| | % control | % control | |
|---|---|---|---|
| | E. coli | Ps. Syr | % control |
| | 5025 | KP71 | Sacc. cer |
| Cellulose-containing | 97 | 100 | 67 |
| microporous superabsorbent | | | |
| composition of Example 1 | | | |
| Silver metallised (AgNP) | 100 | 100 | 100 |
| cellulose-containing | | | |
| microporous superabsorbent | | | |
| composition of Example 2 | | | |
| Gold metallised (AuNP) | 100 | 100 | 90 |
| cellulose-containing | | | |
| microporous superabsorbent | | | |
| composition of Example 3 | | | |

### Example 7—Preparation of Antimicrobial Cellulose-Containing Microporous Superabsorbent Composition Comprising Silver Nanoparticles Utilising UV-Radiation

The cellulose-containing microporous superabsorbent composition of Example 1 0.5 g was evenly distributed over an area to achieve a depth of 1 mm of material within a weighing boat. 5 mL of a 5 mM aqueous solution of AgNO₃ was added to the weighing boat to fully immerse the cellulose-containing microporous superabsorbent composition material. The mixture was mechanically agitated for 5 seconds, then irradiated with UV radiation (395 nm, 9 bulb torch 10 cm from weighing boat) for 4 minutes. The suspension was filtered, the retained solids then dried under a flow of air at 25° C. The dried material was then washed with water three times. Each water wash consisted of suspending the solid material in 10 μmL of water for 10 μminutes at 25° C., followed by isolation of the solid by filtration. The isolated, thrice washed solid material was then dried under a flow of air at 25° C. for 12 hours.

### Example 8—Leaching Test of Antimicrobial Cellulose-Containing Microporous Superabsorbent Composition Comprising Silver Nanoparticles

To establish that the antimicrobial cellulose-containing microporous superabsorbent composition do not exhibit loss of adhered silver nanoparticles on washing, the following test was conducted.

10 g of antimicrobial cellulose-containing microporous superabsorbent composition material was suspended in 4000 μmL of water and 0.5 mL of non-biological detergent. The suspension was stirred for 2 hours at 70° C., filtered through filter paper with a pore size of 5-13 μm, washed 3 times with 500 μmL of water, dried at 60° C. This cycle was repeated 5 times. The wash supernatant analysed by UV-Vis spectroscopy

An antimicrobial cellulose-containing microporous superabsorbent composition comprising silver nanoparticles prepared according to Example 8 was tested. The material did not exhibit any loss of colour or intensity of colour, which is indicative that the silver nanoparticles were retained within the material. As can be seen in FIG. 2, the absence of a peak in the 350-400 nm region in the supernatant confirms no significant loss of silver nanoparticles from the functionalised cellulose active material particles on washing.

### Example 9—Preparation of Simulated Face Mask Material Comprising Cotton and Silver Metallised (AgNP) Cellulose-Containing Microporous Superabsorbent Composition

A simulated face mask material comprising cotton and silver metallised (AgNP) cellulose-containing microporous super absorbent composition was made according to the following process:

A BCI cotton base-sheet (106±3% g/m²) was provided. A Muratex spreading and pressing machine was used to scatter coat the base-sheet with 200 μmg/m² of a mixture. The mixture was solely composed of silver metallised (AgNP) cellulose-containing microporous super absorbent composition prepared according to Example 2 [54 wt. %] and adhesive powder particles (Ecofix Hot-Melt Powder) [46 wt. %]. The scatter coating was realized with a hollow needle that rapidly moved across the cotton base-sheet to provide an even distribution of the powder mixture over the base-sheet. A BCI cotton top-sheet (106±3% g/m²) was then laid on to the base-sheet to enclose the powder between the sheets. The sheets were then passed though hot rollers twice (9 tonnes of pressure equivalent), where the hot rollers applied heat and pressure to melt the adhesive and adhere the base-sheet to the top-sheet around the silver metallised (AgNP) cellulose-containing microporous super absorbent composition particles.

### Example 10—Preparation of TEMPO Cellulose-Containing Microporous Superabsorbent Composition

The cellulose-containing microporous superabsorbent composition prepared according to Example 1 was oxidised by the following method.

The cellulose-containing microporous superabsorbent composition prepared according to Example 1 (10 g of dry solid content), sodium bromide (NaBr, 0.25 g) and TEMPO ((2,2,6,6-Tetramethylpiperidin-1-yl)oxyl, 0.04 g) were mixed together. Then water was added to afford a suspension with a total mass of 2500 g. This suspension was homogenised at 5,000 RPM for a period of 5 minutes. Then, a separate aqueous solution of sodium hypochlorite was prepared (NaClO, 30 g), with the pH adjusted with a 0.5 M aqueous solution of HCl until a pH of 10 was obtained. The first suspension was stirred and the pH monitored using a pH probe. The NaClO was added to the first suspension. The pH of the stirred suspension was maintained in the range of 10.0 to 10.5 by addition of a 0.5 M solution of sodium hydroxide (NaOH) in water for a reaction time of two hours. After the reaction period of two hours, the suspension was filtered and washed until free of NaClO. The solids were isolated and a 1 wt. % suspension in water was prepared. This suspension was passed through a homogenised (Manto Gualin) three times: the first pass at a pressure of 3,000 Bar, the second pass at a pressure of 3,000 Bar and the final pass at 4,500 Bar. The suspension was then left to set, affording a firm gel.

### Example 11—Analysis of Viricidal Activity of Simulated Face Mask Material Comprising Cotton and Silver Metallised (AgNP) Cellulose-Containing Microporous Superabsorbent Composition

The viricidal activity of a simulated face mask material comprising a cotton and silver metallised (AgNP) cellulose microporous superabsorbent composition (prepared according to Example 10) was analysed by adding 100 μl phage Phi6 (DSMZ 21518) suspension (diluted to ^{˜}1e7 cfu/ml) in phage buffer (7 gl⁻¹ Na₂HPO₄, 3 gl⁻¹ KH₂PO₄, 5′gl⁻¹ NaCl, 1 mM MgSO₄·7(H₂O), 1 mM CaCl₂) to 1 cm² of the material for 2 hours at ambient conditions in a 2 ml microtube. Subsequently 1 ml of phage buffer was added, vortexed for 15 seconds and centrifuged for 3 minutes at 1000 g. The supernatant was removed and serially diluted in phage buffer. 100 μl aliquots were placed in 5 ml polypropylene tubes (VWR 211-0049) in triplicate. To each of these, 10 μl of 24 hr old Pseudomonas host cells (DSMZ 21482), which were grown statically at 28° C. in TSB broth (Sigma 22092) supplemented with filter sterilised MgSO₄ (to 5 mM) was added and then incubated for a maximum of 20 μminutes. Subsequently, 3-4 ml of 47° C. molten TSB 0.5% agar supplemented with sterile MgSO₄ to 5 mM was added to each tube. Each tube was then poured on to the surface of petri plates containing 16 mls of solid 1.5% agar TSB plates supplemented with MgSO₄ to 5 mM, before leaving to set for 20 μminutes. Plates were then inverted and incubated at 25° C. for approximately 16 hrs or until visible plaques were observed. Triplicate plates were counted (where the dilution plated yielded between 30 and 300 visible plaques). Triplicate experiments were performed. The material was demonstrated to completely kill phage Phi6.

### Example 12—Functionalisation of TEMPO Cellulose Particulate Material with Proteins

A 1% (w/v) gel of TEMPO oxidised cellulose particulate material (prepared according to Example 1, then oxidised with a TEMPO solution according to example 11) was washed three times, wherein a wash consisted of adding water to the cellulose particulate material slurry to its original volume, vortexing the mixture to resuspend the mixture, centrifuging the mixture at 4200 g for 5 minutes and decanting the supernatant. The cellulose particulate material was washed three additional times with a carbonate buffer (0.1M NaHCO₃ pH 8.3) in the same fashion as above. The washed cellulose particulate material was resuspended in carbonate buffer to obtain a 1.2% stock of functionalised cellulose particulate material.

Proteins and/or enzymes were added to the 1.2% stock of functionalised cellulose particulate material resulting in a mixture comprising 1% (w/v) cellulose particulate material. The mixture was incubated from several hours to up to 60 hours and yielded a stable integration or attachment of the proteins or enzymes to the cellulose particulate material.

The following proteins were added as follows:

Calf intestinal phosphatase (CIP) at a stock concentration of 3000 U/mg (New England Biolabs, M0290S) was added at a concentration of 10 to 200 μg per ml of cellulose particulate material slurry (1% (w/v)). The mixture was incubated for 2 hours at room temperature (about 25° C.) and subsequently stored for future use in a 0.1M pH 8.3 carbonate buffer or phosphate buffered saline (PBS).

Invertase from bakers' yeast at a stock concentration of >300 U/mg (Sigma, 14504) was added at a concentration of 100 μg per ml of cellulose particulate material slurry (1% (w/v)). The mixture was incubated for about 16 hours and subsequently stored for future use in a 0.1M pH 4.5 acetate buffer.

GFP-SpyCatcher was added at a concentration of 25 μg to 2 mg per ml of cellulose particulate material slurry (1% (w/v)). The mixture was incubated for 16 hours or 60 hours and subsequently stored for future use in a 0.1M pH 8.3 carbonate buffer or phosphate buffered saline (PBS).

Dispersin B (DSPB) or Dispersin B containing a metal binding domain (AgDSPB) was added to cellulose particulate material slurry (1% (w/v)) that was not washed or contacted with carbonate buffer, and incubated for several hours.

### Example 13—Functional Attachment of CIP

CIP was attached to functionalised cellulose particulate material according to Example 12 at a concentration of 0, 10, 20 or 40 μg/ml. The mixture was washed and centrifuged twice, after which the supernatants and composition material were assessed for functional attachment of CIP via a BCIP/NBT colorimetric assay. The colorimetric assay is a standard alkaline phosphatase activity assay wherein the substrates 5-bromo-4-chloro-3-indolyl phosphate (BCIP) and nitro blue tetrazolium (NBT) are used to determine the activity of enzymes such as CIP via a colorimetric readout. It can be seen from FIG. 7 that, although some of the CIP has not been attached as is evident from the supernatant colour change, CIP is attached to the cellulose particulate material.

### Example 14—Functional Attachment of Invertase

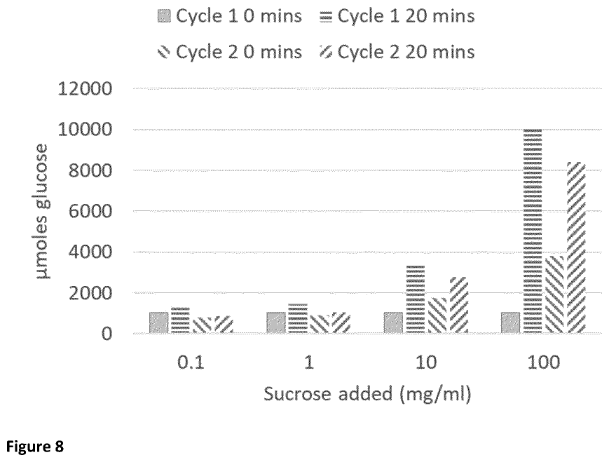

Invertase was attached to functionalised cellulose particulate material according to Example 12. The mixture was then washed three times with water according to Example 12 at a centrifugation of 4122 g and resuspended in water to restore volume equal to the initial volume of TEMPO oxidised cellulose particulate material (1% w/v). The mixture was washed a further three times as with water above, but instead with a 0.1 M pH 4.5 acetate buffer to obtain a 1% (w/v) slurry. To the slurries, different amounts of sucrose was added (100-0.1 mg/ml final volume in reaction volume of 1 ml) and the conversion of sucrose into glucose and fructose by invertase was measured at OD 410 nm according to the manufacturer's protocols, with samples taken at t=0 μminutes and t=20 μminutes. After the first reaction cycle (Cycle 1) the suspension was centrifuged, the supernatant decanted, and resuspended with more sucrose; equivalent to what was initially added. Samples were taken again at t=0 μminutes and t=20 μminutes (Cycle 2). In FIG. 8 it can be seen that sucrose is converted into glucose and fructose thereby confirming the attachment of functional invertase to the cellulose particulate material.

### Example 15—Functional Attachment of GFP-SpyCatcher

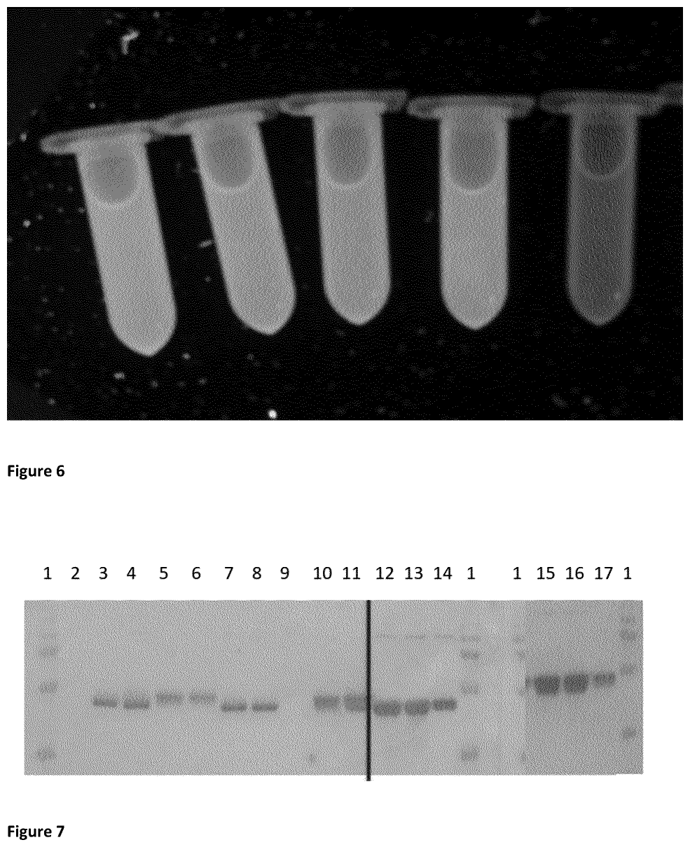

GFP-SpyCatcher was attached to functionalised cellulose particulate material according to Example 12. The mixture was washed several times in PBS to remove unbound components. From FIG. 6 it can be observed that GFP-SpyCatcher is attached to the cellulose particulate material. The SpyCatcher motif contains a Histidine-tag for convenient analysis by for example standard Western Blotting techniques.

### Example 16—Functional Attachment of DSPB and AgDSPB

DSPB or AgDSPB were attached to functionalised cellulose particulate material according to Example 12. The mixture was washed three times in PBS. From FIG. 7 it can be observed that DSPB and AgDSPB can attach robustly to non- and silver-metallised cellulose particulate material. It was found that DSPB can bind at about 1 μg/mm² cellulose particulate material.

### Example 17—Continuous for Preparing an Antimicrobial Cellulose-Containing Microporous Superabsorbent Composition Comprising Silver Nano-Particles (AgNP)

The cellulose-containing microporous superabsorbent composition of Example 1 was deposited onto a moving conveyor belt to form a loose bed of 2-5 mm depth. The loose bed on the conveyor belt was run under a spraying unit, which sprayed the loose bed cellulose-containing microporous superabsorbent composition with 2 weight equivalents of a 5 mM aqueous solution of AgNO₃ at 25° C. The cellulose-containing microporous superabsorbent composition rapidly absorbed the 5 mM aqueous solution of AgNO₃ to afford a loose, dampened bed of bed cellulose-containing microporous composition which has absorbed 2 weight equivalents of a 5 mM aqueous solution of AgNO₃. This loose, dampened bed on the conveyor belt was run under a UV-light source and irradiated with UV light for 30 seconds. The resultant UV-light-irradiated loose bed of silver treated cellulose-containing microporous composition was then dried. The resultant material was analysed and found to comprise silver nanoparticles.

### Example 18—Analysis of Antibacterial Behaviour of Cellulose-Containing Microporous Superabsorbent Composition Comprising [Metal] Nanoparticles with Reductive Virus Structures

8 mm diameter nanocellulose disks were punched from dried thin films of C3PN nanocellulose material prepared from 2% slurry stocks mixed with equal volume of distilled water or 25 mM silver nitrate solution. 5 ml of slurry/water/silver solution mix were pooled on plastic to an area of 4.5 cm diameter and left to dry for 16 hours at 25° C., followed by thrice rinsing in distilled water then allowed to dry. 20 μl of Dispersin B in phosphate buffered saline (PBS) at 0.3 mg/ml or PBS only was applied to the disks, incubated for 16 hours in a damp chamber and then rinsed 3× in 5 ml PBS.

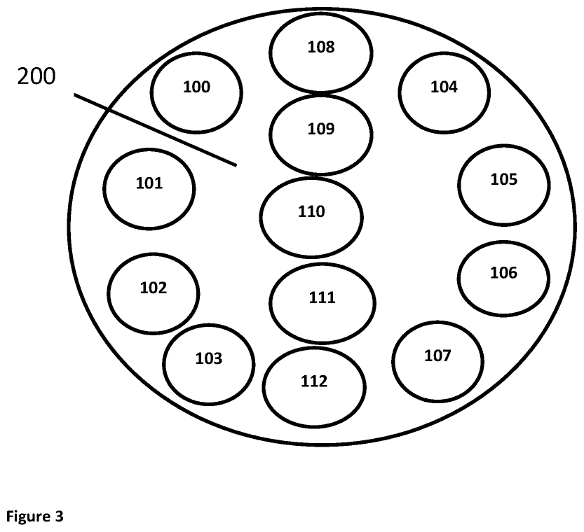

An agar plate (FIGS. 3-5 [200]) was coated with bacteria which express green fluorescent protein. The plate was allowed to develop a lawn of bacteria which express green fluorescent protein (24 hours). The following were deposited onto the lawn:
(1) a disk of silver metallised (AgNP) cellulose-containing microporous superabsorbent composition was treated with 20 μL of water, which was placed onto the bacterial lawn (FIGS. 3-5 [100]);
(2) a disk of silver metallised (AgNP) cellulose-containing microporous superabsorbent composition was treated with 20 μL of phosphate-buffered saline, which was placed onto the bacterial lawn (FIGS. 3-5 [101]);
(3) a disk of silver metallised (AgNP) cellulose-containing microporous superabsorbent composition was treated with 20 μL of Dispersin B (non-silver binding) solution, which was placed onto the bacterial lawn (FIGS. 3-5 [102]);
(4) a disk of silver metallised (AgNP) cellulose-containing microporous superabsorbent composition was treated with 20 μL of Dispersin B (silver binding) solution, which was placed onto the bacterial lawn (FIGS. 3-5 [103]);
(5) a disk of cellulose-containing microporous superabsorbent composition was treated with 20 μL of water, which was placed onto the bacterial lawn (FIGS. 3-5 [104]);
(6) a disk of cellulose-containing microporous superabsorbent composition was treated with 20 μL of phosphate-buffered saline, which was placed onto the bacterial lawn (FIGS. 3-5 [105]); (7) a disk of cellulose-containing microporous superabsorbent composition was treated with 20 μL of Dispersin B (non-silver binding) solution, which was placed onto the bacterial lawn (FIGS. 3-5 [106]);
(8) a disk of cellulose-containing microporous superabsorbent composition was treated with 20 μL of Dispersin B (silver binding) solution, which was placed onto the bacterial lawn (FIGS. 3-5 [107]);
(9) 20 μL of water was directly placed onto the bacterial lawn (FIGS. 3-5 [108]);
(10) 20 μL of phosphate-buffered saline was directly placed onto the bacterial lawn (FIGS. 3-5 [109]);
(11) 20 μL of Dispersin B (non-silver binding) solution was directly placed onto the bacterial lawn (FIGS. 3-5 [110]);
(12) 20 μL of Dispersin B (silver binding) solution was directly placed onto the bacterial lawn (FIGS. 3-5 [111]); and
(13) 20 μL of bleach was directly placed onto the bacterial lawn (FIGS. 3-5 [112]);

Alternatively the disks were applied to agar plates and the plates spread with bacterial culture (500 μl of OD 600 nm 0.2) and again left for 24 hours at 25° C. for Ps. syringae KP71 and 37° C. for E. coli EC5025. After 24 hours contact, the plates were imaged under white light, ultra-violet (UV) light with an orange filter, under UV with the disks of material removed, the disks themselves viewed under UV.

GFP expressing Ps. syringae KP71 and E. coli EC5025 were used as the bacterial test materials. Deposited materials 1-8 were shown to possess significant antibacterial activity. The results are shown in FIGS. 3-5.

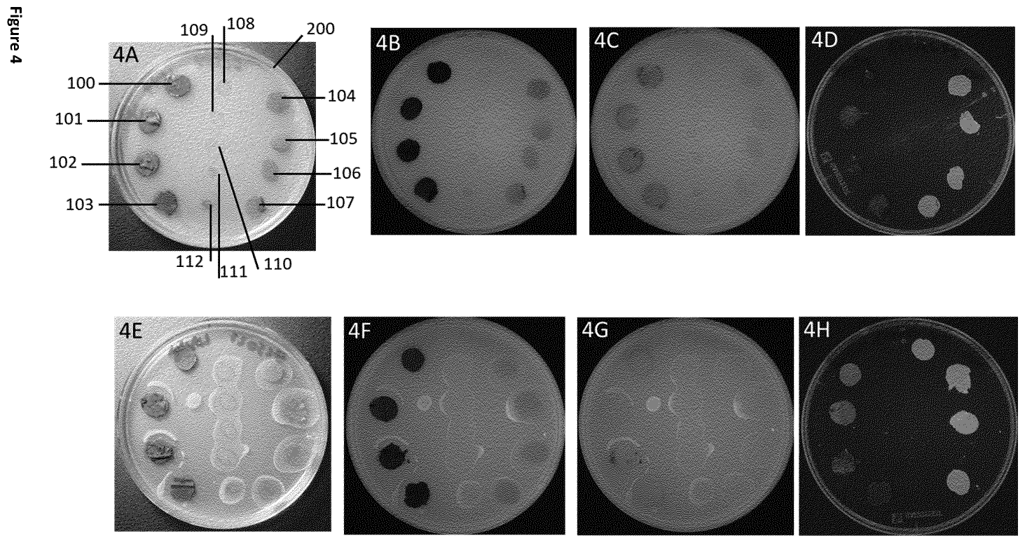

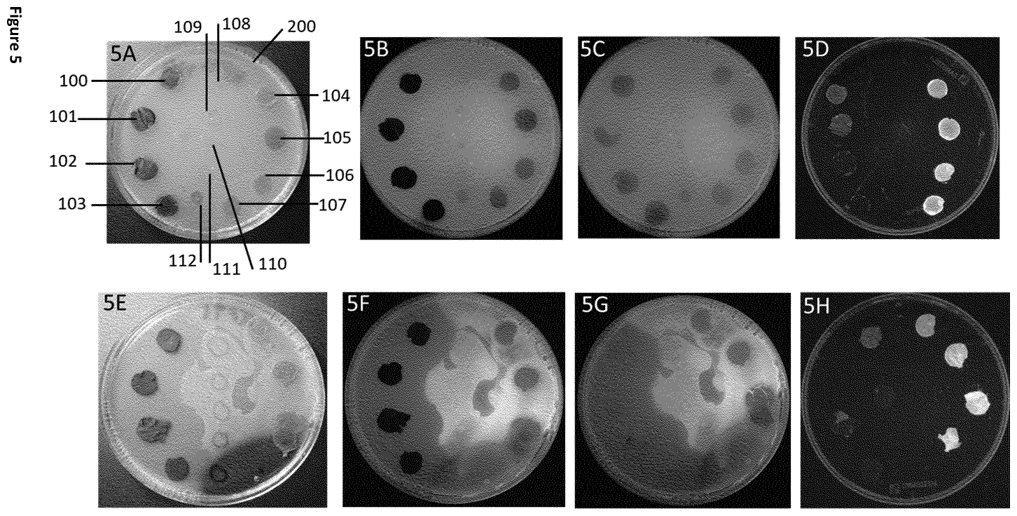

FIG. 3 shows the locations on the plate of each of the 13 deposited materials, which is maintained in the experimental results of FIGS. 4 and 5. FIG. 3 can be used as a general reference for the locations of the deposited materials in FIGS. 4 and 5. For clarity, reference numbers [100]-[112] and [200] are only indicated on FIGS. 4A and 5A, but can be understood to be also present in the other images of FIGS. 4 and 5 on the same general positions.

FIGS. 4A, 4E, 5A and 5E are white light images taken from treated agar plates. FIGS. 4B, 4C, 4D, 4F, 4G, 4H, 5B, 5C, 5D, 5F, 5G and 5H are UV light orange filtered images taken from treated agar plates. Increased pixel intensity (i.e. brighter image) in the UV light images indicates an increased level of GFP, which correlates with a higher number of bacteria. FIG. 4 shows results from the experiments using GFP expressing E. coli EC5025, while FIG. 5 shows results from the experiments using GFP expressing Ps. syringae KP71. FIGS. 4A-D and 5A-D show the experiments as detailed above where the bacteria were allowed to develop a lawn for 24 h after which the materials and disks were deposited. FIGS. 4E-H and 5E-H show the experiments as detailed above where the materials and disks were deposited prior to applying the bacteria. FIGS. 4B, 4F, 5B and 5F show the plates with the disks. FIGS. 4C, 4G, 5C and 5G show the plates with the disks removed. FIGS. 4D, 4H, 5D and 5H show the filter disks after removal from the plates.

As can be clearly seen from the Figures, the silver metallised (AgNP) cellulose-containing microporous superabsorbent composition displayed strong antibacterial properties, whereas the bleach, Dispersin B and unfunctionalized cellulose-containing microporous superabsorbent composition displayed modest antibacterial properties. This is evident when comparing the lower pixel intensity (i.e. lower number of bacteria) of [100], [101], [102] and [103] of FIGS. 4B, 4C, 4D, 4F, 4G, 4H, 5B, 5C, 5D, 5F, 5G and 5H to the higher pixel intensity of the other treated areas. The silver metallised (AgNP) cellulose-containing microporous superabsorbent composition reduced both the number of bacteria on the plates (FIGS. 4C, 4G, 5C and 5G) and had a reduced number of bacteria on the disks (FIGS. 4D, 4H, 5D and 5H) when compared to controls and the non-silver metallised cellulose-containing microporous superabsorbent composition.

Moreover, there was a strong, unexpected synergistic antibacterial function of the combination of silver metallised (AgNP) cellulose-containing microporous superabsorbent composition treated with 20 μL of Dispersin B (both silver binding and non-silver binding) solution (see [102] and [103] of FIGS. 4D, 4H, 5D and 5H), which was strongest for the silver binding Dispersin B solution.

### Example 19—Water Absorption Capacity (WAC)

Water Absorption Capacity (WAC) was determined as follows: Material samples were weighed out in centrifuge tubes, with a minimum sample weight of 0.5 g. Distilled water was added to each sample until it was completely wet. The tubes were then centrifuged at 4000 RPM for 10 μminutes. Following completion of centrifuge, the supernatant was discarded and swollen sample weighed, using the following calculation:

$$WAC = ( Ssw - Sw ) / Sw ,$$

wherein Sw=Sample weight and Ssw=Swollen sample weight.

### Raw Cellulose of Example 1

Sample weight=0.7781
Swollen sample weight=5.2969
WAC=5.81

Generally, the cellulose active material particles according to the invention were found to have a WAC in the range of from 2 to 10.

### Example 3: Antiviral Efficacy

The antiviral activity of the materials were evaluated by the protocol of ISO 18184:2019. Formulations comprising cellulose active material were tested for their viricidal activity against Influenza A virus or Human coronavirus NL63 at a contact time of 2 h relative to a reference control, following ISO18184:2019. The formulations tested were cellulose-containing microporous superabsorbent composition of Example 1. The results are shown in Tables 1 and 2.

TABLE 6
Effect against Influenza A. A value of 2.0 > Mv ≥ 1.0 indicates
good antiviral effect. A value of 3.0 > Mv ≥ 2.0 indicates
very good antiviral effect. A value of Mv ≥ 3.0 indicates excellent antiviral effect.

| Cellulose form vs | | Mv (antiviral | | Affects cell | ISO |
|---|---|---|---|---|---|
| Influenza A [H1N1] | % reduction | activity) | Cytotoxic | sensitivity | compliant |
| Raw cellulose | 99.9 | 3.17 | N | Y | N* |
| (milled, not washed) | | | | | |
| Water processed | 99.9 | 3.63 | N | Y | N |
| Cellulose | | | | | |

*Affected cell susceptibility not valid for ISO18184.

TABLE 7
Effect against Influenza A. A value of 2.0 > Mv ≥ 1.0 indicates
good antiviral effect. A value of 3.0 > Mv ≥ 2.0 indicates
very good antiviral effect. A value of Mv ≥ 3.0 indicates excellent antiviral effect.

| Cellulose form vs Human | % | Mv (antiviral | | Affects cell | |
|---|---|---|---|---|---|
| Coronavirus [NL63] | reduction | activity) | Cytotoxic | sensitivity | ISO compliant |
| Raw cellulose (milled, | 99.9 | 3.53 | N | N | Y |
| not washed) | | | | | |
| Water processed | 99.9 | 3.16 | N | N | Y |
| nanocellulose | | | | | |

As can be seen from Table 6 and 7 all the cellulose materials exhibited good to excellent virucidal activity with a 99.9% reduction in viral titre (both influenza A and human coronavirus) at a contact time of 2 h relative to a reference control, as determined by an ISO18184:2019 procedure. Under these conditions the were ISO compliant except raw and washed cellulose samples since although they had potent antiviral activity, they were found to slightly influence growth of the type of cells used in the influenza assay.

1. A process for preparing an antimicrobial cellulose-containing microporous superabsorbent composition from an herbaceous plant material, the process comprising the step of comminuting dry granulated herbaceous plant material to form microparticles having an average particle diameter of from 100 μm to 800 μm;
to obtain the cellulose-containing microporous superabsorbent composition.

2. The process according to claim 1 comprising the steps of
(a) comminuting dry granulated herbaceous plant material to form microparticles having an average particle diameter of from 100 μm to 800 μm;
(b) optionally contacting the microparticles with an aqueous solution; preferably comprising an alkaline reagent, to wash out soluble components; removing the aqueous solution from the washed microparticles; and also optionally, drying the microparticles,
(c) optionally, forming a film and allowing the film to dry,
(d) at least once contacting the microparticles or the microparticle film with an antimicrobial agent precursor under conditions inductive of the formation, attachment or binding of an antimicrobial agent, and
(e) isolating the antimicrobially modified microporous superabsorbent composition or the antimicrobially modified microporous film.

3. The process according to claim 2, wherein step (d) includes
i) contacting at least a portion of the microparticles, or a film formed from the microparticles according to (a) to (c), with a solution comprising a sufficient quantity of a antimicrobial agent precursor;
ii) maintaining the contact of the microparticles or film with the solution under acceptable conditions for a sufficient period of time to complete the reaction, wherein the reaction comprises forming antimicrobial agent on or in the substrate, attaching the antimicrobial agent to or binding the antimicrobial agent to;
iii) optionally subjecting the mixture to irradiation, preferably by UV light;
iv) rinsing the obtained substrate to remove non-reacted antimicrobial agent precursor and/or reaction products; and,
v) drying the substrate to a desired low moisture content.

4. (canceled)

5. The process according to claim 4, further comprising including a step (d1), comprising the steps of
i) contacting the cellulose particulate material with (2,2,6,6-tetramethylpiperidin-1-yl)oxyl (TEMPO);
ii) optionally contacting the TEMPO-treated cellulose particulate material with an alkaline solution to provide a solution of functionalized cellulose;
iii) optionally drying the solution of functionalized cellulose; and
iv) contacting the cellulose or the functionalized cellulose with the antimicrobial peptides and/or proteins to obtain the antimicrobial peptides and/or protein modified cellulose particulate material.

6. The process according to claim 5 further comprising step i) 1) of washing the cellulose particulate material obtained in i) with water and/or an alkaline solution.

7. The process according to claim 5, wherein the cellulose particulate material is provided in an aqueous solution at a concentration of from 0.1 to 10% (w/v).

8. The process according to claim 5, wherein the alkaline solution is a buffered solution, preferably comprising carbonate and/or an alkali metal, more preferably NaHCO₃, preferably wherein the solution has a pH of from 7 to 13, more preferably of from 7.5 to 9.0, even more preferably of from 8.0 to 8.5.

9. The process according to claim 2, wherein the antimicrobial agent precursor is selected from:
suitable metal nanoparticle pre-cursors, preferably selected from copper salts, silver salts or gold salts, more preferably selected from copper sulfate (CuSO₄), copper acetate (Cu(OAc)₂), silver nitrate (AgNO₃) and the chlorides of gold, even more preferably selected from copper sulfate (CuSO₄), silver nitrate (AgNO₃) and chloroauric acid (HAuCl₄) and most preferably selected from silver nitrate (AgNO₃); and
one or more antimicrobial peptides or proteins, preferably wherein the antimicrobial peptides or proteins comprise peptides or proteins having an amino acid sequence selected from the group consisting of AU1, AU2, AU3, 1037, LF1-II, KR12, lactoferrampin, FK-16 and Dispersin B (SEQ ID NOs 1 to 9), most preferably Dispersin B (SEQ ID NO 9).

10. The process according to claim 5, comprising following a TEMPO-treated cellulose particulate material with a metallizing treatment, to obtain a multiply modified composition comprising antimicrobial peptide and metal modification.

11. The process according to claim 10, further comprising at least one of:
contacting the composition or the peptides or proteins with a solution or suspension comprising a suitable metal nanoparticle pre-cursor, preferably wherein the solution or suspension is alkaline, preferably the alkaline solution or suspension is buffered, preferably comprising carbonate and/or an alkali metal, more preferably NaHCO₃, and preferably having a pH of from 7 to 13, more preferably of from 7.5 to 9.0, even more preferably of from 8.0 to 8.5; and
retaining the TEMPO-treated cellulose particulate material during contacting or washing in a container comprising openings of a size and dimension that prevents a substantial portion of the cellulose particulate material from passing through.

12.-13. (canceled)

14. The process according to claim 2, wherein the rinsing is performed with an aqueous solution, and additionally comprising the step of dewatering the substrate after the rinsing step.

15. A cellulosic superabsorbent material comprising a fluid-adsorbent volume area obtained according to the process of claim 1, comprising one or more of a biologically and/or chemically active antimicrobial agent(s), and configured to render the material antimicrobial when exposed to a microbial agent.

16. A method of treating fluids, preferably aqueous fluids, including menses, bodily fluids, skin, cosmetic compositions, or wound exudates, the method comprising contacting the fluid with the material of claim 15.

17. The material according to claim 15:
wherein the material is shaped into, or comprised in, a wound dressing, a sanitary pad, a tampon, an absorbent dressing, a diaper, a sponge, a sanitary wipe, an isolation and surgical gown, a glove, a surgical scrub, sutures, sterile packaging, a floor mat, a burn dressing, a mattress cover, bedding, soft furnishings, curtains, clothing, an air filter for vehicles, planes, buildings or generally for HVAC systems, a water filter, military protective garment, a face mask, a device for protection against biohazards and biological warfare agents, lumber, paper, cardboard, meat or fish packaging material, paints and coatings, apparel for food handling, and other surfaces or materials required to exhibit a non-leaching antimicrobial property and to release over time portions of biologically or chemically active compounds; or
comprising all or part of a wound dressing, sanitary pad, a tampon, an intrinsically antimicrobial absorbent dressing, a diaper, toilet paper, a sponge, a sanitary wipe, food preparation surfaces, gowns, gloves, surgical scrubs, sutures, needles, sterile packings, floor mats, lamp handle covers, burn dressings, gauze rolls, blood transfer tubing or storage container, mattress cover, bedding, soft furnishing, curtain, clothing, sheet, towel, underwear, socks, cotton swabs, applicators, exam table covers, head covers, cast liners, splint, paddings, lab coats, air filters for vehicles, planes or HVAC systems, water filters, military protective garments, face masks, devices for protection against biohazards and biological warfare agents, lumber, meat packaging material, paper currency, powders, and other surfaces required to exhibit an essentially non-leaching antimicrobial or enhanced microbial pathogen binding properties, and to release over time portions of the biologically or chemically active compound.

18. (canceled)

19. An inherently antimicrobial composition obtainable by the process according to claim 2, comprising:
a) an herbaceous cellulose-containing substrate; and
b) an antimicrobial agent adhered to the substrate, wherein the antimicrobial agent substrate, or surface area exhibits antimicrobial activity due to the presence of the antimicrobial agent.

20. The composition of claim 19, wherein the material comprises all or part of a wound dressing, sanitary pad, a tampon, an intrinsically antimicrobial absorbent dressing, a diaper, toilet paper, a sponge, a sanitary wipe, food preparation surfaces, gowns, gloves, surgical scrubs, sutures, needles, sterile packings, floor mats, lamp handle covers, burn dressings, gauze rolls, blood transfer tubing or storage container, mattress cover, bedding, soft furnishings, curtains, clothing, sheet, towel, underwear, socks, cotton swabs, applicators, exam table coves, head covers, cast liners, splint, paddings, lab coats, air filters for vehicles such as automobiles, trains, boats or planes, air filters for architectural HVAC systems, water filters, military protective garments, face masks, devices for protection against biohazards and biological warfare agents, lumber, meat packaging material, paper currency, powders, water filters, and other surfaces required to exhibit a non-leaching antimicrobial or enhanced dye binding properties, and to release over time portions of the biologically or chemically active compound.

21. A method for for the retention and/or destruction of microbes or viruses, the method comprising contacting a microbe or virus with the composition of claim 19.

22. The method of claim 21, wherein the composition comprises at least one of:
a plurality of biologically and/or chemically active compounds selected from the group consisting of: antibiotics, analgesics, anti-inflammatories, strong oxidizing agents, matrix metalloproteinase inhibitors, proteins, peptides, and fungicidal compounds;
metal and/or metal oxide material attached to the cellulose, preferably wherein the metal and/or metal oxides comprise copper, silver or gold, more preferably silver;
antimicrobial peptides or proteins, preferably wherein the antimicrobial peptides or proteins comprise peptides or proteins having an amino acid sequence selected from the group consisting of AU1, AU2, AU3, 1037, LF1-II, KR12, lactoferrampin, FK-16 and Dispersin B (SEQ ID NOs 1 to 9), most preferably Dispersin B (SEQ ID NO 9); and
peptides or proteins comprising metal binding and/or metal reducing peptides, preferably further comprising metal and/or metal oxide material attached to the peptides or proteins.

23.-25. (canceled)

26. The composition according to claim 22, wherein the peptides or proteins are antibacterial, antifungal, antiparasitic or antiviral, preferably antibacterial.

27. A method of treating or covering a wound, comprising contacting the wound with a substrate comprising the antimicrobially modified absorbent materials according to claim 22.


SEQUENCE LISTING (11 sequences)

SEQ ID NO: 1 - protein, 10 aa, synthetic construct
SEKLFFGASL

SEQ ID NO: 2 - protein, 10 aa, synthetic construct
SEKLWWGASL

SEQ ID NO: 3 - protein, 10 aa, synthetic construct
GASLWWSEKL

SEQ ID NO: 4 - protein, 9 aa, synthetic construct
KRFRIRVRV

SEQ ID NO: 5 - protein, 11 aa, synthetic construct
  REGION 1..11: note = part of human lactoferrin structural component
GRRRSVQWCAV

SEQ ID NO: 6 - protein, 12 aa, Homo sapiens
KRIVQRIKDFLR

SEQ ID NO: 7 - protein, 17 aa, synthetic construct
  REGION 1..17: note = part of human lactoferrin structural component
WNLLRQAQEKFGKDKSP

SEQ ID NO: 8 - protein, 16 aa, Homo sapiens
FKRIVQRIKDFLRNLV

SEQ ID NO: 9 - protein, 361 aa, Aggregatibacter actinomycetemcomitans
NCCVKGNSIYPQKTSTKQTGLMLDIARHFYSPEVIKSFIDTISLSGGNFLHLHFSDHENYAIESHLLNQRAENAVQGKDGIYINPYTGKPFLSYRQLDDIKAYAKAKGIELIPELDSPNHMTAIFKLVQKDRGVKYLQGLKSRQVDDEIDITNADSITFMQSLMSEVIDIFGDTSQHFHIGGDEFGYSVESNHEFITYANKLSYFLEKKGLKTRMWNDGLIKNTFEQINPNIEITYWSYDGDTQDKNEAAERRDMRVSLPELLAKGFTVLNYNSYYLYIVPKASPTFSQDAAFAAKDVIKNWDLGVWDGRNTKNRVQNTHEIAGAALSIWGEDAKALKDETIQKNTKSLLEAVIHKTNGDE

SEQ ID NO: 10 - protein, 16 aa, synthetic construct
  MOD_RES 16: note = amide
  REGION 1..16: note = Modified synthetic antimicrobial fragment FK-16 of human cathelicidin (LL-37)
FKRIVQRIKDFLRNLV

SEQ ID NO: 11 - protein, 9 aa, synthetic construct
  MOD_RES 9: note = amine
KRFRIRVRV